(19) United States
(12) Patent Application Publication
(10) Pub. No.: US 2024/0108652 A1
(43) Pub. Date: Apr. 4, 2024

(54) ENHANCING METABOLIC FITNESS OF T CELLS TO TREAT CANCER

(71) Applicant: University of Florida Research Foundation, Incorporated, Gainesville, FL (US)

(72) Inventors: Loic Pierre DELEYROLLE, Gainesville, FL (US); Duane MITCHELL, Gainesville, FL (US)

(21) Appl. No.: 18/263,008

(22) PCT Filed: Feb. 16, 2022
(86) PCT No.: PCT/US2022/016573

§ 371 Date: Jul. 26, 2023

Related U.S. Application Data
(60) Provisional application No. 63/150,635, filed on Feb. 18, 2021.

(51) Int. Cl. A61K 35/17; A61K 39/00; A61P 35/00; C07K 14/705; C12N 5/0783

(52) U.S. Cl. CPC A61K 35/17; A61K 39/4611; A61K 39/464402; A61P 35/00; C07K 14/705; C12N 5/0636; A61K 2239/47

(57) ABSTRACT

Described are modified T cells overexpressing one or more glucose transporters. Pharmaceutical compositions containing the glucose transporter overexpressing T cells are also described. The glucose transporter overexpressing T cells and pharmaceutical compositions can be used in adoptive cell therapies.

### CROSS-REFERENCE TO RELATED APPLICATIONS

This application is a national phase filing of International Application No. PCT/US2022/016573, filed Feb. 16, 2022, which claims the benefit of U.S. Provisional Application No. 63/150,635, filed Feb. 18, 2021, the content of each is incorporated herein by reference.

### SEQUENCE LISTING

The Sequence Listing written in file 574346_SeqListing.txt is 21 kilobytes in size, was created Feb. 4, 2022, and is hereby incorporated by reference.

### BACKGROUND

Adoptive T cell transfer (ACT) has emerged as a viable therapeutic to treat cancer. While promising, the efficacy of this approach is often limited by a complex immunosuppressive tumor microenvironment. These complexities mean that more sophisticated T cell products are necessary for the treatment of malignancies. T cell functions are metabolically regulated and undergo metabolic reprogramming upon activation marked by an increased need for glucose to support their greater bioenergetic and biosynthetic needs. Cancerous tumors, including brain tumors, are supported by a microenvironment characterized by tumor-imposed metabolic restrictions with fierce nutrient competition, especially for glucose. In the tumor microenvironment, tumor cells impose glucose restriction to T cells, therefore mediating their hypo-responsiveness resulting in immune evasion.

Improved ACT therapeutics are needed to treat cancer.

### SUMMARY

Described are therapeutic modalities based on reprogramming the metabolic qualities of anti-tumor immune cells to enhance immunotherapy. Specifically, we describe methods to improve T cell glycolysis by glucose transporter over-expression, providing a competitive advantage leading to greater survival, tumor infiltration and anti-tumor activity. This therapeutic modality based on tumor metabolism and immunometabolism concepts can be applied to any proliferative diseases such as cancer.

GLUT-overexpressing T cells are described that express one or more heterologous SCL2A nucleic acids. The GLUT-overexpressing T cells overexpress one or more glucose transporters. The glucose transporter can be, but is not limited to: GLUT1, GLUT 2, GLUT3, GLUT4, GLUT5 GLUT6, GLUT7, GLUT8, GLUT9, GLUT10, GLUT11, GLUT12, GLUT13, and GLUT 14.

T cells can be modified to produce GLUT-overexpressing T cells by introducing one or more heterologous SCL2A nucleic acids into the T cell. The SCL2A nucleic acids can be introduced into the T cells by methods known in the art for introducing a nucleic acid into a T cell. Such methods include, but are not limited to, transfection of a DNA vector, transfection of an RNA vector, viral vectors, retroviral vectors, or a CRISPR-Cas system.

In some embodiments, increasing expression of a glucose transporter in a T cell comprising contacting the T cell with a hormone, wherein the hormone causes increased expression of an endogenous glucose transporter in the T cell. The hormone can be, but is not limited to, an insulin, a testosterone, a glucocorticoid, or a retinoic acid.

In some embodiments, increasing expression of a glucose transporter in a T cell comprising expressing a heterologous nucleic acid encoding an insulin receptor, a CD28, an IL2-R, an IL7-R, or an IL3-R in the T cell, wherein expression of the insulin receptor, the CD28, the IL2-R, the IL7-R, or the IL3-R in the T cell results in increased expression of one or more endogenous glucose transporters in the T cell or increased translocation of one or more endogenous glucose transporters to the plasma membrane of the T cell.

The T cell that can be modified to produce a GLUT-overexpressing T cell is not limited to any particular type of T cell of source of the T cells. The T cell can be, but is not limited to: a primary T cell, a culture T cell, a autologous T cell, an allogeneic T cell, a T cell obtained from bone marrow, a T cell obtained from a lymph node, a T cell obtained from a thymus, a tumor infiltrating lymphocyte, a T cell obtained from a spleen, a T cell from umbilical cord blood, a universal allogenic T cell, a universal CAR T cell, a CAR T cell, a naïve T cell, an effector T cell, an effector memory T cell, a CD4+/CD8+ T cell, a helper T cell, a CD4+ T cell, a CD4+ helper T cell, a Th1 T cell, a Th2 T cell, a cytotoxic T cell, a CD8+ T cell, peripheral blood mononuclear cell (PBMC), a peripheral blood leukocyte (PBL), a memory T cell, a central memory T cell, a regulatory T cell, an αβ T cell, a γδ T cell, a modified T cell, a T cell for use in adoptive cell transfer therapy, a TCR-engineered T cell, a chimeric antigen receptor (CAR) T cell, a first generation CAR T cell, a second generation CAR T cell, a third generation CAR T cell, a fourth generation CAR T cell, dual-antigen receptor CAR T cell, or a CAR T cell having an inducible suicide gene, or a combination thereof.

A T cell can be further modified prior, concurrent with, at subsequent to modifying the T cell to overproduce a GLUT protein. For example, the T cell can be further modified to express a T cell receptor (TCR), an αβ TCR, a γδ TCR, a CAR, a first generation CAR, a second generation CAR, a third generation CAR, a fourth generation CAR, a secreted cytokine, a cytokine receptor, a chimeric cytokine receptor, a CD40L, a4-1BBL, a dominant-negative TGF-β receptor II, a constitutively active Akt, an antibody-like protein, a nanobody, or a bispecific T-cell engager. A T cell can also be modified introducing a tumor mRNA, total tumor mRNA, slow cycling cancer cell mRNA, or cancer stem cell mRNA into the T cell.

Any of the described GLUT-overexpressing T cells can be provided in a pharmaceutical composition. The pharmaceutical compositions can contain, for example, a pharmaceutically acceptable excipient, one or more additional active pharmaceutical ingredients, or one or more T cells that haven't been modified to overproduce a GLUT protein.

Any of the described GLUT-overexpressing T cells can be provided in a population of T cells. The population can be, but is not limited to: an essentially clonal population of T cells derived from a single GLUT-overexpressing T cell, a substantially homogenous population of GLUT-overexpressing T cells, a population of T cells comprising T cells in which all or substantially all of the T cells in the population are modified to overexpress a GLUT gene, or a population of T cells in which less than all of the T cells are modified to overexpress the GLUT gene. The population can be homogenous or heterogeneous with respect to type of T cell.

The GLUT-overexpressing T cells have increase metabolic fitness. The GLUT-overexpressing T cells exhibit one or more of: increased survival, increased growth, increased differentiation, increased activation, increased glucose utilization, increased glucose uptake, increase fitness, increased metabolic fitness, increased tumor infiltration, or increased anti-tumor activity in low glucose or glucose-restricted conditions compared to a genetically similar T cell that does not overexpress a GLUT gene when measured under the same conditions.

Also described are methods of treating cancer in a subject by administering to the subject any of the described GLUT-overexpressing T cells, or populations or pharmaceutical compositions containing any of the described GLUT-overexpressing T cells. In some embodiments, the methods comprise: obtaining a T cell from the subject or obtaining a donor T cell, modifying the T cell to overexpress one or more heterologous glucose transporters thereby forming a GLUT-overexpressing T cell, and administering the GLUT-overexpressing T cell to the subject. In some embodiments, the methods further comprise further modifying the T cell. The further modification can be done before, concurrent with, or after modifying the T cell to overexpress one or more glucose transporters. In some embodiments, the methods further comprise isolating or purifying the T cells. In some embodiments, the methods further comprise expanding the T cells to produce a population of T cells.

In some embodiments, any of the described GLUT-overexpressing T cells, or populations or pharmaceutical compositions containing any of the described GLUT-overexpressing T cells can be used in adoptive cell transfer therapy.

Further embodiments of the invention provide related populations of T cells, pharmaceutical compositions, and methods of treating a disease.

### BRIEF DESCRIPTION OF THE DRAWINGS

FIG. 1. Graphs illustrating effective GBM cells on glucose uptake by T cells. (A) FACS sorting showing competition of glucose as measure by 2NBDG. (B) Line graph showing glucose uptake by T cells as a function of GBM cell to T cell ratio.

FIG. 2. Graphs illustrating effect of glucose concentration on IFNγ production by T cells.

FIG. 3. (A) Micrographs illustrating retrovirally-induced expression of GLUT1 or GLUT3 in T cells. T cells were transfected with an expression vector encoding for GFP and GLUT1 or GFP and GLUT3. (B) FACS sorting showing expression of GLUT1 and GFP in modified T cells. (C) Graph illustrating increased expression of GLUT3 in modified T cells.

FIG. 4. Graph illustrating increased consumption of glucose by T cells overexpressing GLUT1 or GLUT3 compared to T cells expressing GFP. Glucose in the media was quantitated. Less glucose in the media correlates with increased glucose consumption by the T cells.

FIG. 5. Graphs illustrating viability (A), differentiation (B), and activation (C) of GLUT-overexpressing T cells in non-restricted glucose.

FIG. 6. Graph illustrating increased survival of GLUT-overexpressing T cells in glucose-restricted conditions.

FIG. 7. Graphs illustrating increased differentiation (A) and activation (B) of GLUT-overexpressing T cells in glucose-restricted conditions. T cells were activated with Kluc RNA electroporated dendritic cells, but the T cells were not antigen-specific.

FIG. 8. Graphs illustrating expression of GLUT1 in human T cells.

FIG. 9. Graphs illustrating (A) increased glucose consumption, (B) survival/expansion, and (C) activation of human T cells overexpressing GLUT1 or control T cells in varying concentrations of glucose.

FIG. 10. Graphs illustrating tumor size as determined by fluorescence (A) and levels of GLUT1 overexpressing TILs, GLUT3 overexpressing TILs, and control TILs (B).

FIG. 11. Graph illustrating activation of GLUT1 overexpressing CD70 CAR T cells, GLUT3 overexpressing CD70 CAR T cells, or control CD70 CAR-T cells in various concentrations of glucose (*=p<0.05, **=p<0.005, one-way ANOVA).

FIG. 12. Graphs illustrating increased localization of GLUT1 overexpressing CD70 CAR T cells (CAR-G1) compared to control CD70 CAR T cells (CAR-GFP) (Top panel), and increased anti-tumor activity of GLUT1 overexpressing CD70 CAR T cells (CAR-G1) compared to control CD70 CAR T cells (CAR-GFP) (lower panel) and control T cells (GFP) (lower panel).

### DETAILED DESCRIPTION

### A. Definitions

Before describing the present teachings in detail, it is to be understood that the disclosure is not limited to specific compositions or process steps, as such may vary. It should be noted that, as used in this specification and the appended claims, the singular form “a,” “an,” and “the” include plural references unless the context clearly dictates otherwise. Thus, for example, reference to “a drug” includes a plurality of drugs and the like. The conjunction “or” is to be interpreted in the inclusive sense, i.e., as equivalent to “and/or,” unless the inclusive sense would be unreasonable in the context.

In general, the term “about” indicates variation in a quantity of a component of a composition not having any significant effect on the activity or stability of the composition. When the specification discloses a specific value for a parameter, the specification should be understood as alternatively disclosing the parameter at “about” that value. All ranges are to be interpreted as encompassing the endpoints in the absence of express exclusions, such as “not including the endpoints”; thus, for example, “within 10-15” or “from 10 to 15” includes the values 10 and 15. Also, the use of “comprise,” “comprises,” “comprising,” “contain,” “contains,” “containing,” “include,” “includes,” and “including” are not intended to be limiting. It is to be understood that both the foregoing general description and detailed description are exemplary and explanatory only and are not restrictive of the teachings. To the extent that any material incorporated by reference is inconsistent with the express content of this disclosure, the express content controls.

Unless specifically noted, embodiments in the specification that recite “comprising” various components are also contemplated as “consisting of” or “consisting essentially of” the recited components. Embodiments in the specification that recite “consisting essentially of” various components are also contemplated as “consisting of” “Consisting essentially of” means that additional component(s), composition(s) or method step(s) that do not materially change the basic and novel characteristics of the compositions and methods described herein may be included in those compositions or methods.

A “nucleic acid” includes both RNA and DNA. RNA and DNA include, but are not limited to, cDNA, genomic DNA, plasmid DNA, synthetic RNA or DNA, and mRNA. A nucleic acid can be formulated with a delivery agent such as, but not limited, a cationic lipid, a peptide, a cationic polymer, or a virus. Nucleic acid also includes modified RNA or DNA.

An “expression vector” refers to a nucleic acid (e.g., RNA or DNA) encoding one or more expression products (e.g., peptide (i.e., polypeptide or protein)). An expression vector may be, but is not limited to, a virus or attenuated virus (viral vector), a plasmid, a linear DNA molecule, an mRNA, a CRISPR RNA, a CISPR system, or a composition comprising the nucleic acid encoding the expression product. An expression vector is capable of expressing one or more polypeptides in a cell, such a mammalian cell. The expression vector may comprise one or more sequences necessary for expression of the encoded expression product. A variety of sequences can be incorporated into an expression vector to alter expression of the coding sequence. The expression vector may comprise one or more of: a 5′ untranslated region (5′ UTR), an enhancer, a promoter, an intron, a 3′ untranslated region (3′ UTR), a terminator, and a polyA signal operably linked to the DNA coding sequence. A vector may also comprise one or more sequences that alter stability of a messenger RNA (mRNA), RNA processing, or efficiency of translation. Any of the described nucleic acids encoding a GLUT protein can be part of an expression vector designed to express the GLUT protein in a cell.

A viral vector can be, but is not limited to, an AAV vector, an adenovirus, a retrovirus, a gammaretrovirus, a lentivirus, a vaccinia virus, an alphavirus, or a herpesvirus. An adeno-associated virus can be, but is not limited to, AAV1, AAV2, AAV2/1, AAV2/2, AAV2/4, AAV2/5, AAV2/6, AAV2/7, AAV2/8, AAV2/9, AAV4, AAV5, AAV6, AAV7, AAV7m8, AAV8, AAV9, and AAV44. Nucleic acid encoding the desired protein can be packaged into the viral vectors using methods and constructs known in the art. Vectors can be manufactured in large scale quantities and/or in high yield. Vectors can be manufactured using GMP manufacturing. Vectors are can be formulated to be safe and effective for injection into a mammalian subject. Vectors can be delivery to a cell, a subject, an organ or tissue in the subject, or cells in a subject using methods known in the art.

The term “CRISPR RNA (crRNA)” has been described in the art (e.g., in Makarova et al. (2011) Nat Rev Microbiol 9:467-477; Makarova et al. (2011) Biol Direct 6:38; Bhaya et al. (2011) Annu Rev Genet 45:273-297; Barrangou et al. (2012) Annu Rev Food Sci Technol 3:143-162; Jinek et al. (2012) Science 337:816-821; Cong et al. (2013) Science 339:819-823; Mali et al. (2013) Science 339: 823-826; and Hwang et al. (2013) Nature Biotechnol 31:227-229). A crRNA contains a sequence (spacer sequence or guide sequence) that hybridizes to a target sequence in the genome. A target sequence can be any sequence that is unique compared to the rest of the genome and is adjacent to a protospacer-adjacent motif (PAM).

A “protospacer-adjacent motif” (PAM) is a short sequence recognized by the CRISPR complex. The precise sequence and length requirements for the PAM differ depending on the CRISPR system used, but PAMs are typically 2-5 base pair sequences adjacent the protospacer (i.e., target sequence). Non-limiting examples of PAMs include NGG, NNGRRT, NN[A/C/T]RRT, NGAN, NGCG, NGAG, NGNG, NGC, and NGA.

A “trans-activating CRISPR RNA” (tracrRNA) is an RNA species that facilitates binding of the RNA-guided DNA endonuclease (e.g., Cas) to the guide RNA.

A “CRISPR system” comprises a guide RNA, either as a crRNA and a tracrRNA (dual guide RNA) or an sgRNA, and RNA-guided DNA endonuclease. The guide RNA directs sequence-specific binding of the RNA-guided DNA endonuclease to a target sequence. In some embodiments, the RNA-guided DNA endonuclease contains a nuclear localization sequence. In some embodiments, the CRISPR system further comprises one or more fluorescent proteins and/or one or more endosomal escape agents. In some embodiments, the gRNA and RNA-guided DNA endonuclease are provided in a complex. In some embodiments, the gRNA and RNA-guided DNA endonuclease are provided in one or more expression constructs (CRISPR constructs) encoding the gRNA and the RNA-guided DNA endonuclease. Delivery of the CRISPR construct(s) to a cell results in expression of the gRNA and RNA-guided DNA endonuclease in the cell. The CRISPR system can be, but is not limited to, a CRISPR class 1 system, a CRISPR class 2 system, a CRISPR/Cas system, a CRISPR/Cas9 system, a CRISPR/zCas9 system and a CRISPR/Cas3 system.

The term “plasmid” refers to a nucleic acid that includes at least one sequence encoding a polypeptide (e.g., an expression vector) that is capable of being expressed in a mammalian cell. A plasmid can be a closed circular DNA molecule. A variety of sequences can be incorporated into a plasmid to alter expression of the coding sequence or to facilitate replication of the plasmid in a cell. Sequences can be used that influence transcription, stability of a messenger RNA (mRNA), RNA processing, or efficiency of translation. Such sequences include, but are not limited to, 5′ untranslated region (5′ UTR), promoter, introns, and 3′ untranslated region (3′ UTR). In some embodiments, plasmids can be transformed into bacteria, such as E. coli.

A “promoter” is a DNA regulatory region capable of binding an RNA polymerase in a cell (e.g., directly or through other promoter-bound proteins or substances) and initiating transcription of a coding sequence. A promoter may comprise one or more additional regions or elements that influence transcription initiation rate, including, but not limited to, enhancers. A promoter can be, but is not limited to, a constitutively active promoter, a conditional promoter, an inducible promoter, or a cell-type specific promoter. Examples of promoters can be found, for example, in WO 2013/176772. The promoter can be, but is not limited to, CMV promoter, Igκ promoter, mPGK, SV40 promoter, β-actin promoter (such as, but not limited to a human or chicken β-actin promoter), α-actin promoter, SRα promoter, herpes thymidine kinase promoter, herpes simplex virus (HSV) promoter, mouse mammary tumor virus long terminal repeat (LTR) promoter, adenovirus major late promoter (Ad MLP), rous sarcoma virus (RSV) promoter, and EFla promoter. The CMV promoter can be, but is not limited to, CMV immediate early promoter, human CMV promoter, mouse CNV promoter, and simian CMV promoter. The promoter can also be a hybrid promoter. Hybrid promoters include, but are not limited to, CAG promoter.

“Operable linkage” or being “operably linked” refers to the juxtaposition of two or more components (e.g., a promoter and another sequence element) such that both components function normally and allow the possibility that at least one of the components can mediate a function that is exerted upon at least one of the other components. For example, a promoter can be operably linked to a coding sequence if the promoter controls the level of transcription of the coding sequence in response to the presence or absence of one or more transcriptional regulatory factors. Operable linkage can include such sequences being contiguous with each other or acting in trans (e.g., a regulatory sequence can act at a distance to control transcription of the coding sequence).

A “heterologous” sequence is a sequence which is not normally present in a cell, genome, or gene in the genetic context in which the sequence is currently found. A heterologous sequence can be a sequence derived from the same gene and/or cell type, but introduced into the cell or a similar cell in a different context, such as on an expression vector or in a different chromosomal location or with a different promoter. A heterologous sequence can be a sequence derived from a different gene or species than a reference gene or species. A heterologous sequence can be from a homologous gene from a different species, from a different gene in the same species, or from a different gene from a different species. For example, a regulatory sequence may be heterologous in that it is linked to a different coding sequence relative to the native regulatory sequence.

The term “cancer” includes a myriad of diseases generally characterized by inappropriate cellular proliferation, abnormal or excessive cellular proliferation. Examples of cancer include, but are not limited to, breast cancer, triple negative breast cancer, colon cancer, prostate cancer, pancreatic cancer, melanoma, lung cancer, ovarian cancer, kidney cancer, brain cancer, or sarcomas.

The “tumor microenvironment” refers to the environment in and around a tumor and may include the non-malignant vascular and stromal tissue that aid in growth and/or survival of a tumor, such as by providing the tumor with oxygen, growth factors, and nutrients, or inhibiting immune response to the tumor. A tumor microenvironment includes the cellular environment in which the tumor exists, including surrounding blood vessels, immune cells, fibroblasts, bone marrow-derived inflammatory cells, lymphocytes, signaling molecules and the extracellular matrix.

A “homologous” sequence (e.g., nucleic acid sequence or amino acid sequence) refers to a sequence that is either identical or substantially similar to a known reference sequence, such that it is, for example, at least 75%, at least 80%, at least 85%, at least 90%, at least 95%, at least 96%, at least 97%, at least 98%, at least 99%, or 100% identical to the known reference sequence. Sequence identity can be determined by aligning sequences using algorithms, such as BESTFIT, FASTA, and TFASTA in the Wisconsin Genetics Software Package Release 7.0, Genetics Computer Group, 575 Science Dr., Madison, Wis.), using default gap parameters, or by inspection, and the best alignment (i.e., resulting in the highest percentage of sequence similarity over a comparison window). Percentage of sequence identity is calculated by comparing two optimally aligned sequences over a window of comparison, determining the number of positions at which the identical residues occurs in both sequences to yield the number of matched positions, dividing the number of matched positions by the total number of matched and mismatched positions not counting gaps in the window of comparison (i.e., the window size), and multiplying the result by 100 to yield the percentage of sequence identity. Unless otherwise indicated the window of comparison between two sequences is defined by the entire length of the shorter of the two sequences. Homologous sequences can include, for example, orthologs (orthologous sequences) and paralogs (paralogous sequences). Homologous genes, for example, typically descend from a common ancestral DNA sequence, either through a speciation event (orthologous genes) or a genetic duplication event (paralogous genes). “Orthologous” genes are genes in different species that evolved from a common ancestral gene by speciation. Orthologs typically retain the same function in the course of evolution. “Paralogous” genes include genes related by duplication within a genome. Paralogs can evolve new functions in the course of evolution.

An “active ingredient” is any component of a drug product intended to furnish pharmacological activity or other direct effect in the diagnosis, cure, mitigation, treatment, or prevention of disease, or to affect the structure or any function of the body of humans or other animals. Active ingredients include those components of the product that may undergo chemical change during the manufacture of the drug product and be present in the drug product in a modified form intended to furnish the specified activity or effect. A dosage form for a pharmaceutical contains the active pharmaceutical ingredient, which is the drug substance itself, and excipients, which are the ingredients of the tablet, or the liquid in which the active agent is suspended, or other material that is pharmaceutically inert. During formulation development, the excipients can be selected so that the active ingredient can reach the target site in the body at the desired rate and extent.

A “pharmacologically effective amount,” “therapeutically effective amount,” or simply “effective amount” refers to that amount (dose) of a described active pharmaceutical ingredient or pharmaceutical composition to produce the intended pharmacological, therapeutic, or preventive result. An “effective amount” can also refer to the amount of, for example an excipient, in a pharmaceutical composition that is sufficient to achieve the desired property of the composition. An effective amount can be administered in one or more administrations, applications, or dosages.

As used herein, “dose,” “unit dose,” or “dosage” can refer to physically discrete units suitable for use in a subject, each unit containing a predetermined quantity of an active pharmaceutical ingredient and/or a pharmaceutical composition thereof calculated to produce the desired response or responses in association with its administration.

The terms “treat,” “treatment,” and the like, mean the methods or steps taken to provide relief from or alleviation of the number, severity, and/or frequency of one or more symptoms of a disease or condition in a subject. Treating generally refers to obtaining a desired pharmacological and/or physiological effect. The effect can be, but does not necessarily have to be, prophylactic in terms of preventing or partially preventing a disease, symptom or condition thereof. The effect can be therapeutic in terms of a partial or complete cure of a disease, condition, symptom or adverse effect attributed to the disease, disorder, or condition. The term treatment can include: (a) preventing the disease from occurring in a subject who may be predisposed to the disease but has not yet been diagnosed as having it; (b) inhibiting the disease, i.e., arresting its development; and (c) relieving the disease, i.e., mitigating or ameliorating the disease and/or its symptoms or conditions. Treating can refer to both therapeutic treatment alone, prophylactic treatment alone, or both therapeutic and prophylactic treatment. Those in need of treatment (subjects in need thereof) can include those previously diagnosed with cancer. Treating can include inhibiting the disease, disorder or condition, e.g., impeding its progress; and relieving the disease, disorder, or condition, e.g., causing regression of the disease, disorder and/or condition. Treating the disease, disorder, or condition can include ameliorating at least one symptom of the particular disease, disorder, or condition, even if the underlying pathophysiology is not affected.

“Adoptive cell therapy,” also termed Adoptive Cell Transfer or Adoptive T Cell Transfer, is a type of immunotherapy in which T cells are given to a patient to help the body fight diseases, such as cancer. In some embodiments, T cells are taken from the patient's own blood or tumor tissue, grown in large numbers in the laboratory, and then given back to the patient to help the immune system fight the cancer. In some embodiments, T cells are taken from a donor, grown in large numbers in the laboratory, and then given back to the patient to help the immune system fight the cancer. Sometimes, the T cells are changed in the laboratory to make them better able to target the patient's cancer cells and kill them. Types of adoptive cell transfer include chimeric antigen receptor T-cell (CAR T-cell) therapy and tumor-infiltrating lymphocyte (TIL) therapy. Adoptive cell transfer is also called adoptive cell therapy, cellular adoptive immunotherapy, and T-cell transfer therapy.

Chimeric antigen receptor T-cell (CAR T-cell) therapy is a type of treatment in which a patient's T cells are changed in the laboratory so they will attack cancer cells. T cells are taken from a patient's blood. Then the gene for a receptor that binds to a certain protein on the patient's cancer cells is added to the T cells in the laboratory. This receptor is called a chimeric antigen receptor (CAR). Large numbers of the CAR T cells are grown in the laboratory and given to the patient, such as by infusion.

### B. GLUT-Overexpressing T Cells

Tumor cells undergo metabolic reprogramming to enhance their glycolytic flux to support exponential growth. A result of this tumor metabolic shift there is an increase of tumor glucose uptake and utilization. This increase in tumor glucose uptake and utilization creates a glucose restricted microenvironment in and around the tumor.

Quiescent T cells (resting naïve T cells and TM cells) primarily use oxidative phosphorylation to generate energy in the form of ATP. However, upon activation, T cells upregulate glycolysis. This switch to aerobic glycolysis by activated T cells results in a significant increase in glucose consumption. Glucose is essential for T cell survival, activation and expansion.

By increasing glucose uptake and creating a local environment that is glucose restricted, cancer cells compete with T cells for glucose, thereby contributing to T cell hyporesponsiveness in the tumor and tumor immune evasion. This T cell hyporesponsiveness in a glucose restricted tumor can be overcome by increasing the metabolic fitness of T cells.

Described are GLUT-overexpressing T cells and compositions, including pharmaceutical compositions, and formulations comprising the GLUT-overexpressing T cells. The GLUT-overexpressing T cells and compositions and formulations comprising the GLUT-overexpressing T cells can be used in Adoptive T Cell Transfer, such as to treat cancer. Methods of using the GLUT-overexpressing T cells and compositions and formulations comprising the GLUT-overexpressing T cells to treat a subject having a cancerous tumor, such as a solid cancerous tumor are also described.

In some embodiment, a GLUT-overexpressing T cell comprises a heterologous SCL2A nucleic acid sequence the provides for expression of a GLUT protein. A GLUT-overexpressing T cell expressed the GLUT protein at a level that is higher than the level of the GLUT protein expressed by a similar T cell that has not been modified with respect to GLUT expression (e.g., wild-type T cells, or a T cell that does not contain the heterologous SCL2A nucleic acid sequence). For example, if the T cell has been modified to comprise a heterologous SCL2A1 nucleic acid sequence, as described in more detail below, the modified T cell comprising the heterologous SCL2A1 nucleic acid sequence expresses GLUT1 at a level that is higher than the level of GLUT1 expressed by a control T cell that does not contain the heterologous SCL2A1 nucleic acid sequence.

T cells which have not been modified to overexpress a GLUT gene may express a basic level of GLUT mRNA or protein. However, T cells overexpressing the GLUT gene express more GLUT mRNA or protein as compared to a control T cell that has not been modified to overexpress the GLUT gene. In some embodiments, the GLUT-overexpressing T cell or GLUT-overexpressing T cell population expresses the GLUT mRNA or protein at a level that is at least 25% more, at least 50% more, at least 75% more, at least twice (100% more), at least 125% more, at least 150% more, or at least three times more than a control T cell or control T cell population that has not been modified to overexpress the GLUT gene when measured under the same conditions. In some embodiments, the GLUT-overexpressing T cell or T cell population expresses 4× or more, 5× or more, 6× or more, 7× or more, 8× or more, 9× or more, or 10× or more of the GLUT mRNA or protein as compared to a control T cell or T cell population that has not been modified to overexpress the GLUT gene when measure under the same conditions. Any suitable method known in the art can be utilized to determine the amount of GLUT mRNA or polypeptide present in a T cell or a population thereof.

Described are methods of modifying T cells comprising introducing into one or more T cells, an expression vector encoding a GLUT protein, wherein introducing the expression vector encoding the GLUT protein into the T cell results in overexpression of the GLUT protein by the T cell.

GLUT-overexpressing T cells can also be generated by inducing overexpression of an endogenous glucose transporter in the T cells or by increasing translocation of an endogenous glucose transporter to the plasma membrane in the T cells. In some embodiments, increasing expression of a glucose transporter in a T cell comprising contacting the T cell with a hormone, wherein the hormone causes increased expression of an endogenous glucose transporter in the T cell. The hormone can be, but is not limited to, an insulin, a testosterone, a glucocorticoid, or a retinoic acid. In some embodiments, increasing expression of an endogenous glucose transporter in a T cell comprising expressing a heterologous nucleic acid encoding an insulin receptor, a CD28, an IL2-R, an IL7-R, or an IL3-R in the T cell, wherein expression of the insulin receptor, the CD28, the IL2-R, the IL7-R, or the IL3-R in the T cell results in increased expression of one or more endogenous glucose transporters in the T cell or increased translocation of one or more endogenous glucose transporters to the plasma membrane of the T cell.

The GLUT-overexpressing T cells are useful in the treatment of cancer. The cancer can be a single solid tumor and a metastatic cancer.

The T cell can be any T cell. The T cell may be, but is not limited to, a cultured T cell, a primary T cell, a T cell from a cultured T cell line, or a T cell obtained from a mammal. A T cell from a cultured T cell line includes, but is not limited to, a Jurkat T cell and a SupT1. A primary T cell can be from the subject (autologous T cell) to be treated or a donor subject (allogeneic T cell).

The T cell can be sourced from a mammal. The T cell can be, but is not limited to, a T cell obtained from blood, a T cell obtained from bone marrow, a T cell obtained from a lymph node, a T cell obtained from a thymus, a tumor infiltrating lymphocyte (TIL), a T cell obtained from a spleen, or a T cell from umbilical cord blood, each of which can be from an autologous donor source or an allogeneic donor source. The T cell can also be a universal allogenic T cell (e.g., induced pluripotent stem cells (iPSCs), HSCs) or a universal CAR T cell. In some embodiments, the T cell is a human T cell. In some embodiments, the T cell is obtained from a human subject.

The T cell can be any type of T cell. The T cell can, but is not limited to, a naïve T cell (T_{naïve} cell), an effector T cell, an effector memory T cell (Tₑₘ cell), a CD4+/CD8+ T cell, a helper T cell, a CD4+ T cell, a CD4+ helper T cell, a Th1 T cell, a Th2 T cell, a cytotoxic T cell, a CD8+ T cell, peripheral blood mononuclear cell (PBMC), a peripheral blood leukocyte (PBL), a tumor infiltrating T cell (TIL), a memory T cell, a central memory T cell (T_{cm} cell), a regulatory T cell, an αβ T cell, a γδ T cell, a modified T cell, a T cell for use in adoptive cell transfer therapy (e.g., adoptive T cell transfer therapy), a TCR-engineered T cell, a chimeric antigen receptor (CAR) T cell, a first generation CAR T cell (CAR having a single signaling domain, such as a CD3ζ signaling domain), a second generation CAR T cell (CAR having a co-stimulatory domain), a third generation CAR T cell (CAR having multiple co-stimulatory domains), a fourth generation CAR T cell (TRUCK or armored CAR), dual-antigen receptor CAR T cell, or a CAR T cell having an inducible suicide gene, or a combination thereof. In some embodiments, the T cell has been previously modified, e.g., a CART cell. The T cell can be a single T cell or a population of T cells. In some embodiments, the population of T cells is a substantially homogenous population with respect to the type of T cell (e.g., the population of T cells may be CD4⁺/CD8⁺ T cells, cytotoxic T cells, or CART cells). In some embodiments, the population of T cells is a heterogenous population with respect to the type of T cell (e.g., comprising cytotoxic T cells and helper T cells). In some embodiments, the population of T cells is an essentially clonal population of T cells.

The T cell may be isolated or purified from a source using any suitable technique known in the art for isolating or purifying T cells. The T cell (population) can be expanded prior to modification to overexpress a GLUT protein, or after modification to overexpress a GLUT protein. The T cells can be isolated, enriched, or purified prior to and/or after modification to overexpress a GLUT gene. Expansion of the T cell population can be done using any available method in the art for expanding T cells.

The T cells are modified to produce GLUT-overexpressing T cells by introducing a heterologous nucleic acid sequence (i.e., a heterologous SCL2A nucleic acid) encoding and expressing a GLUT (glucose transporter) protein. Glucose transporters are a group of membrane proteins that facilitate transport of glucose across the plasma membrane. The GLUT proteins are encoded by the SLC2A genes. The GLUT or SLC2A family are a protein family that is found in most mammalian cells. There are 14 members of the glucose transporter (GLUT) family (Table 1). Of the 14 GLUT genes, GLUT1, GLUT3, GLUT6, and GLUT8 are the most active in endogenous T cells. GLUT1 and GLUT3 are the best characterized, with GLUT3 having the highest affinity for glucose.

The GLUT protein can be, but is not limited to, any of the GLUT proteins in Table 1. In some embodiments, the GLUT protein is GLUT1. In some embodiments, the GLUT protein is GLUT3. The GLUT protein can be encoding by a GLUT coding sequence in any of the corresponding SLC2A genes in Table 1. A heterologous SCL2A nucleic acid may be a recombinant nucleic acid. The nucleic acid encoding the GLUT coding sequence can comprise one or more regulatory sequences to facilitate expression of the GLUT protein in the T cell. Regulatory sequences include, but are not limited to, enhancers, promoters, transcription initiation sequences, translation initiation sequences, termination codons, 5′ untranslated regions (UTR) sequences, 3′ UTR sequences, transcription terminal signals, and polyA sequences. The GLUT coding sequence can be operably linked to its native regulatory sequence(s) or one or more heterologous regulatory sequences. The promoter can be any promoter that is active in T cells. The promoter can be a native GLUT gene promoter or a heterologous promoter. The promoter can be a non-viral promoter or a viral promoter. Exemplary viral promoters include, but are not limited to, a cytomegalovirus (CMV) promoter, an SV40 promoter, an RSV promoter, or a promoter found in the long-terminal repeat of the murine stem cell virus. The promoter can be a constitutive promoter or an inducible promoter.

The heterologous SCL2A nucleic acid may comprise any suitable SLC2A nucleotide sequence which encodes any suitable GLUT amino acid sequence. In some embodiments, the SLC2A nucleotide sequence comprises a SLC2A1 sequence. In some embodiments, the SLC2A nucleotide sequence comprises a SLC2A3 sequence. In some embodiments, the GLUT amino acid sequence comprises a GLUT1 amino acid sequence. In some embodiments, the GLUT amino acid sequence comprises a GLUT3 amino acid sequence.

In some embodiments, a SCL2A nucleic acid sequence is modified to increase expression in a T cell or to add a sequence or modify a sequence to improve intracellular targeting of the encoded GLUT protein, such as to direct the GLUT protein to the plasma membrane.

TABLE 1
GLUT proteins and encoding genes.

| | | GenBank Accession No. |
|---|---|---|
| Protein | Gene | (variant) |
| GLUT1 | SLC2A1 | NM_006516 |
| GLUT2 | SLC2A2 | NM_000340 |
| GLUT3 | SLC2A3 | NM_006931 |
| GLUT4 | SLC2A4 | NM_001042 |
| GLUT5 | SLC2A5 | NM_003039 |
| GLUT6 | SLC2A6 | NM_017585 |
| GLUT7 | SLC2A7 | NM_207420 |
| GLUT8 | SLC2A8 | NM_014580 |
| GLUT9 | SLC2A9 | NM_020041 |
| | | (NM_001001290) |
| GLUT10 | SLC2A10 | NM_030777 |
| GLUT11 | SLC2A11 | NM_030807 |
| | | (NM_001024938) |
| | | (NM_001024939) |
| GLUT12 | SLC2A12 | NM_145176 |
| GLUT13 | SLC2A13 | NM_052885 |
| GLUT14 | SLC2A14 | NM_153449 |

The SLC2A1 nucleotide sequence can include, but is not limited to, SEQ ID NO: 1, 5, or 7, or an ortholog thereof. The GLUT1 amino acid sequence can include, but is not limited to SEQ ID NO: 2 or an ortholog thereof.

In some embodiments, the SLC2A1 nucleic acid comprises a nucleic acid sequence having at least 70%, at least 80%, at least 90%, at least 95%, at least 97%, at least 98%, at least 99%, or 100% identity to SEQ ID NO: 1, 5, or 7, wherein the encoded polypeptide retains the functional activity of GLUT1. In some embodiments, the SLC2A1 nucleic acid comprises a nucleic acid encoding a polypeptide having at least 70%, at least 80%, at least 90%, at least 95%, at least 97%, at least 98%, at least 99%, or 100% amino acid sequence identity to SEQ ID NO: 2, wherein the encoded polypeptide retains the functional activity of GLUT1. In some embodiments, the SLC2A1 nucleic acid comprises a nucleic acid encoding a polypeptide having at least 70%, at least 80%, at least 90%, at least 95%, at least 97%, at least 98%, at least 99%, or 100% amino acid sequence identity to SEQ ID NO: 2, wherein the encoded polypeptide has glucose transport activity.

The SLC2A3 nucleotide sequence can include, but is not limited to SEQ ID NO: 3, 6, or 8 or an ortholog thereof. The GLUT3 amino acid sequence can include, but is not limited to SEQ ID NO: 4 or an ortholog thereof.

In some embodiments, the SLC2A3 nucleic acid comprises a nucleic acid sequence having at least 70%, at least 80%, at least 90%, and least 95%, at least 97%, at least 98%, at least 99%, or 100% identity to SEQ ID NO: 3, 6, or 8, wherein the encoded polypeptide retains the functional activity of GLUT3. In some embodiments, the SLC2A3 nucleic acid comprises a nucleic acid encoding a polypeptide having at least 70%, at least 80%, at least 90%, and least 95%, at least 97%, at least 98%, at least 99%, or 100% amino acid sequence identity to SEQ ID NO: 4, wherein the encoded polypeptide retains the functional activity of GLUT3. In some embodiments, the SLC2A3 nucleic acid comprises a nucleic acid encoding a polypeptide having at least 70%, at least 80%, at least 90%, and least 95%, at least 97%, at least 98%, at least 99%, or 100% amino acid sequence identity to SEQ ID NO: 4, wherein the encoded polypeptide has glucose transport activity.

In some embodiments, the SLC2A1 or SLC2A3 nucleic acid is operably linked to a promoter.

TABLE 2
Sequences.

SLC2A1 (SEQ ID NO: 1):
atggagcccagcagcaagaagctgacgggtcgcctcatgctggccgtgggaggagcagtgcttggc
tccctgcagtttggctacaacactggagtcatcaatgccccccagaaggtgatcgaggagttctac
aaccagacatgggtccaccgctatggggagagcatcctgcccaccacgctcaccacgctctggtcc
ctctcagtggccatcttttctgttgggggcatgattggctccttctctgtgggccttttcgttaac
cgctttggccggcggaattcaatgctgatgatgaacctgctggccttcgtgtccgccgtgctcatg
ggcttctcgaaactgggcaagtcctttgagatgctgatcctgggccgcttcatcatcggtgtgtac
tgcggcctgaccacaggcttcgtgcccatgtatgtgggtgaagtgtcacccacagcccttcgtggg
gccctgggcaccctgcaccagctgggcatcgtcgtcggcatcctcatcgcccaggtgttcggcctg
gactccatcatgggcaacaaggacctgtggcccctgctgctgagcatcatcttcatcccggccctg
ctgcagtgcatcgtgctgcccttctgccccgagagtccccgcttcctgctcatcaaccgcaacgag
gagaaccgggccaagagtgtgctaaagaagctgcgcgggacagctgacgtgacccatgacctgcag
gagatgaaggaagagagtcggcagatgatgcgggagaagaaggtcaccatcctggagctgttccgc
tcccccgcctaccgccagcccatcctcatcgctgtggtgctgcagctgtcccagcagctgtctggc
atcaacgctgtcttctattactccacgagcatcttcgagaaggcgggggtgcagcagcctgtgtat
gccaccattggctccggtatcgtcaacacggccttcactgtcgtgtcgctgtttgtggtggagcga
gcaggccggcggaccctgcacctcataggcctcgctggcatggcgggttgtgccatactcatgacc
atcgcgctagcactgctggagcagctaccctggatgtcctatctgagcatcgtggccatctttggc
tttgtggccttctttgaagtgggtcctggccccatcccatggttcatcgtggctgaactcttcagc
cagggtccacgtccagctgccattgccgttgcaggcttctccaactggacctcaaatttcattgtg
ggcatgtgcttccagtatgtggagcaactgtgtggtccctacgtcttcatcatcttcactgtgctc
ctggttctgttcttcatcttcacctacttcaaagttcctgagactaaaggccggaccttcgatgag
atcgcttccggcttccggcaggggggagccagccaaagtgacaagacacccgaggagctgttccat
cccctgggggctgattcccaagtg
SLC2A1 (SEQ ID NO: 5):
atggagcccagcagcaagaagctgaccggcagactgatgctggctgttggcggagctgttctgggc
tctctgcagttcggctacaacaccggcgtgatcaacgcccctcagaaagtgatcgaggaattctac
aaccagacctgggtgcacagatacggcgagagcatcctgcctaccacactgacaacactgtggtcc
ctgagcgtggccatcttttctgtcggcggcatgatcggcagcttcagcgtgggcctgttcgtgaac
agattcggcagacggaactccatgctgatgatgaacctgctggccttcgtgtccgccgtgctgatg
ggcttttctaagctgggcaagagcttcgagatgctgatcctgggaagattcatcatcggcgtgtac
tgcggcctgaccaccggctttgtgcctatgtacgtgggcgaagtgtcccctacagctctgagaggt
gctctgggaacactgcaccagctgggcatcgttgtgggaatcctgatcgcccaggtgttcggcctg
gatagcatcatgggcaacaaggacctgtggcctctgctgctgagcatcatcttcatccccgctctg
ctgcagtgcatcgtgctgcctttctgtcctgagagccccagattcctgctgatcaaccggaacgag
gaaaaccgggccaagagcgtgctgaagaaactgagaggcaccgccgacgtgacccacgacctgcaa
gagatgaaggaagagtccagacagatgatgcgcgagaagaaagtgaccatcctggaactgttcaga
agccccgcctacagacagcccatcctgattgctgtggtgctgcagctgagccagcagctgtctgga
atcaacgccgtgttctactacagcaccagcatcttcgagaaggccggcgtgcagcagcctgtgtat
gccacaatcggcagcggcatcgtgaacaccgcctttaccgtggtgtccctgtttgtggtggaaaga
gccggcagaagaaccctgcacctgattggactggccggcatggccggatgtgccattctgatgaca
atcgccctggctctgctggaacagctgccctggatgagctacctgagcatcgtggccattttcggc
ttcgtggccttctttgaagtcggccctggacctattccttggtttatcgtggccgagctgttttct
cagggccccagacctgctgctattgccgtggccggattcagcaactggaccagcaatttcatcgtg
ggcatgtgcttccagtacgtcgagcagctgtgtggcccctacgtgttcatcattttcaccgtgctg
ctggtcctgttcttcatcttcacctacttcaaggtgcccgagacaaagggcagaaccttcgacgag
atcgccagcggctttagacaaggcggcgctagccagagcgataagacccctgaggaactgtttcac
cctctgggcgccgattctcaggtgtga
mouse SLC2A1 (SEQ ID NO: 7):
atggatcccagcagcaagaaggtgacgggccgcctcatgttggctgtgggaggagcagtgctcgga
tcactgcagttcggctataacactggtgtcatcaacgccccccagaaggttattgaggagttctac
aatcaaacatggaaccaccgctacggagagcccatcccatccaccacactcaccacgctttggtct
ctctccgtggccatcttctctgtcgggggcatgattggttccttctctgtcggcctctttgttaat
cgctttggcaggcggaactccatgctgatgatgaacctgttggcctttgtggctgctgtgcttatg
ggcttctccaaactgggcaagtcctttgagatgctgatcctgggccgcttcatcatcggtgtgtac
tgcggcctgactactggctttgtgcccatgtatgtgggagaggtgtcacctacagctctacgtgga
gccctaggcacactgcaccagctgggaatcgtcgttggcatccttattgcccaggtgtttggctta
gactccatcatgggcaatgcagacttgtggcctctgctgctcagtgtcatcttcatcccagccctg
ctacagtgtatcctgttgcccttctgccccgagagcccccgcttcctgctcatcaatcgtaacgag
gagaaccgggccaagagtgtgctgaagaagcttcgagggacagccgatgtgacccgagacctgcag
gagatgaaagaagagggtcggcagatgatgcgggagaagaaggtcaccatcttggagctgttccgc
tcacccgcctaccgccagcccatcctcatcgctgtggtgctgcagctgtcccagcagctgtcgggt
atcaatgctgtgttctactactcaacgagcatcttcgagaaggcaggtgtgcagcagcctgtgtac
gccaccatcggctccggtatcgtcaacacggccttcactgtggtgtcgctgtttgttgtagagcga
gctggacgacggaccctgcacctcattggcctggctggcatggcaggctgtgctgtgctcatgacc
atcgccctggccttgctggaacggctgccttggatgtcctatctgagcatcgtggccatctttggc
tttgtggccttctttgaagtaggccctggtcctattccatggttcattgtggccgagctgttcagc
caggggccccgtcctgctgctattgctgtggctggcttctccaactggacctcaaacttcattgtg
ggcatgtgcttccagtatgtggagcaactgtgcggcccctacgtcttcatcatcttcacggtgctc
ctcgtgctcttcttcatcttcacctacttcaaagtccctgagaccaaaggccgaaccttcgatgag
atcgcttccggcttccggcaggggggtgccagccaaagtgacaagacacccgaggagctcttccac
cctctgggggcggactcccaagtgtga
GLUTI (SEQ ID NO: 2):
MEPSSKKLTGRLMLAVGGAVLGSLQFGYNTGVINAPQKVIEEFYNQTWVHRYGESILPTTLTTLWS
LSVAIFSVGGMIGSFSVGLFVNRFGRRNSMLMMNLLAFVSAVLMGFSKLGKSFEMLILGRFIIGVY
CGLTTGFVPMYVGEVSPTALRGALGTLHQLGIVVGILIAQVFGLDSIMGNKDLWPLLLSIIFIPAL
LQCIVLPFCPESPRFLLINRNEENRAKSVLKKLRGTADVTHDLQEMKEESRQMMREKKVTILELFR
SPAYRQPILIAVVLQLSQQLSGINAVFYYSTSIFEKAGVQQPVYATIGSGIVNTAFTVVSLFVVER
AGRRTLHLIGLAGMAGCAILMTIALALLEQLPWMSYLSIVAIFGFVAFFEVGPGPIPWFIVAELFS
QGPRPAAIAVAGFSNWTSNFIVGMCFQYVEQLCGPYVFIIFTVLLVLFFIFTYFKVPETKGRTFDE
IASGFRQGGASQSDKTPEELFHPLGADSQV
SLC2A3 (SEQ ID NO: 3):
atggggacacagaaggtcaccccagctctgatatttgccatcacagttgctacaatcggctctttc
caatttggctacaacactggggtcatcaatgctcctgagaagatcataaaggaatttatcaataaa
actttgacggacaagggaaatgccccaccctctgaggtgctgctcacgtctctctggtccttgtct
gtggccatattttccgtcgggggtatgatcggctccttttccgtcggactcttcgtcaaccgcttt
ggcaggcgcaattcaatgctgattgtcaacctgttggctgtcactggtggctgctttatgggactg
tgtaaagtagctaagtcggttgaaatgctgatcctgggtcgcttggttattggcctcttctgcgga
ctctgcacaggttttgtgcccatgtacattggagagatctcgcctactgccctgcggggtgccttt
ggcactctcaaccagctgggcatcgttgttggaattctggtggcccagatctttggtctggaattc
atccttgggtctgaagagctatggccgctgctactgggttttaccatccttcctgctatcctacaa
agtgcagcccttccattttgccctgaaagtcccagatttttgctcattaacagaaaagaagaggag
aatgctaagcagatcctccagcggttgtggggcacccaggatgtatcccaagacatccaggagatg
aaagatgagagtgcaaggatgtcacaagaaaagcaagtcaccgtgctagagctctttagagtgtcc
agctaccgacagcccatcatcatttccattgtgctccagctctctcagcagctctctgggatcaat
gctgtgttctattactcaacaggaatcttcaaggatgcaggtgttcaagagcccatctatgccacc
atcggcgcgggtgtggttaatactatcttcactgtagtttctctatttctggtggaaagggcagga
agaaggactctgcatatgataggccttggagggatggctttttgttccacgctcatgactgtttct
ttgttattaaaggataactataatgggatgagctttgtctgtattggggctatcttggtctttgta
gccttctttgaaattggaccaggccccattccctggtttattgtggccgaactcttcagccagggc
ccccgcccagctgcgatggcagtggccggctgctccaactggacctccaacttcctagtcggattg
ctcttcccctccgctgctcactatttaggagcctacgtttttattatcttcaccggcttcctcatt
accttcttggcttttaccttcttcaaagtccctgagacccgtggcaggacttttgaggatatcaca
cgggcctttgaagggcaggcacacggtgcagatagatctggaaaggacggcgtcatggagatgaac
agcatcgagcctgctaaggagaccaccaccaatgtc
SLC2A3 (SEQ ID NO: 6):
atggggacccagaaagtgacccctgctctgatcttcgccatcaccgtggccaccatcggcagcttc
cagttcggctataacaccggcgtgatcaacgcccctgagaagatcatcaaagagttcatcaacaag
accctgaccgacaagggcaacgccccaccttctgaagtgctgctgacaagcctgtggtccctgagc
gtggccatcttttctgtcggcggcatgatcggaagcttcagcgtgggcctgttcgtgaacagattc
ggcagacggaactccatgctgatcgtgaacctgctggctgtgaccggcggctgttttatgggactg
tgcaaggtggccaagagcgtggaaatgctgatcctgggcagactggtcatcggcctgttctgtggc
ctgtgcaccggcttcgtgcctatgtacatcggcgagatcagccccacagctctgagaggcgctttc
ggcacactgaaccagctgggcatcgttgtgggaatcctggtggcccagatcttcggcctggaattc
atcctgggctccgaggaactgtggcctctgctgctgggctttacaatcctgcctgccatcctgcag
tctgccgctctgcctttctgtcctgagagccccagattcctgctgatcaaccggaaagaggaagag
aacgccaagcagatcctgcagcggctgtggggaacacaggatgtgtcccaggacatccaagagatg
aaggacgagagcgcccggatgagccaagagaagcaagtgaccgtgctggaactgttccgggtgtcc
agctacagacagcccatcatcatcagcatcgtgctgcagctgagccagcagctgtccggaatcaac
gccgtgttctactacagcaccggcatcttcaaggacgccggggtgcaagagcctatctacgccaca
attggagccggcgtcgtgaacaccatctttaccgtggtgtccctgttcctggtggaaagagctggg
agaagaaccctgcacatgatcggccttggcggaatggccttctgcagcaccctgatgacagtgtcc
ctgctgctgaaggacaactacaacggcatgagcttcgtgtgcatcggcgccatcctggtgttcgtg
gccttctttgagatcggccctggacctattccttggtttatcgtggccgagctgttctctcagggc
cctagacctgctgctatggccgtggccggatgtagcaactggaccagcaatttcctcgtgggactg
ctgttccctagcgccgctcattatctgggcgcctacgtgttcatcatcttcaccggctttctgatc
accttcctggccttcaccttcttcaaggtgcccgagacacggggcagaaccttcgaggatatcacc
cgggcctttgaaggacaggctcacggcgctgatagaagcggaaaggacggcgtgatggaaatgaac
agcatcgagcccgccaaagaaaccaccaccaacgtgtga
mouse SLC2A3 (SEQ ID NO: 8):
atggggacaacgaaggtgaccccatctctggtgttcgccgtgactgttgccacgatcggctctttc
cagtttggctacaacactggagtcatcaatgcacctgagacaatcctaaaggactttcttaactac
actttggaagagcggttagaagacctaccaagtgagggactgctgactgccctctggtccttatgt
gtggccatcttctctgttggtggcatgattggctctttttctgttggactctttgtcaaccgcttt
ggcagacgcaactctatgcttctagtcaacttgctggccatcattgcgggctgccttatgggattc
gccaagatagcggagtctgttgaaatgctgatcctgggccgcttactcattggcattttctgtggc
ctgtgcacgggctttgtgcctatgtacattggagaggtgtctcccactgcccttcggggtgcattt
ggcacactaaaccagctgggcatcgttgttgggattctggtagctcagatctttggtttggacttt
attctgggctctgaagaactgtggcctgggctccttggcttaaccatcattccagctatcctgcaa
agcgcagcccttccattttgccctgagagtccaagattcttgctcattaacaaaaaggaggaagac
caagctacagagatcctgcagcgcttgtggggcacctcggacgtggtccaggagatccaggagatg
aaggatgagagtgttcggatgtcacaggagaagcaggtgactgtgctggagctcttcaggtcaccc
aactacgtccagccgcttctcatctccattgtcctccagctgtctcagcagctctctgggatcaat
gctgtgttctattactcaacaggaatcttcaaggacgcgggtgtccaggaaccgatctatgccacg
attggagcaggcgtggtcaatactatcttcactgtagtttctctgttcctggtggagagggcaggg
aggagaaccctgcatatgataggcctgggaggcatggctgtttgctccgttttcatgacgatttcg
ctgttactaaaggatgactatgaagccatgagctttgtctgtattgtggctatcttgatctacgta
gccttctttgagattggacctggccccattccctggtttattgtggctgagctcttcagccagggc
ccccgcccagctgccattgcggtggctggctgttgtaactggacctccaactttctggtcggaatg
ctcttcccctcagctgcagcctacttaggagcctacgtttttatcatcttcgctgccttcctcatc
ttcttcctaatcttcaccttcttcaaagtcccggagaccaaaggcaggactttcgaggacattgcc
cgggccttcgaggggcaggcgcactctggaaaaggccctgccggtgtggagttgaacagcatgcag
ccggtcaaggagacccctggcaacgcctga
GLUT3 (SEQ ID NO: 4):
MGTQKVTPALIFAITVATIGSFQFGYNTGVINAPEKIIKEFINKTLTDKGNAPPSEVLLTSLWSLS
VAIFSVGGMIGSFSVGLFVNRFGRRNSMLIVNLLAVTGGCFMGLCKVAKSVEMLILGRLVIGLFCG
LCTGFVPMYIGEISPTALRGAFGTLNQLGIVVGILVAQIFGLEFILGSEELWPLLLGFTILPAILQ
SAALPFCPESPRELLINRKEEENAKQILQRLWGTQDVSQDIQEMKDESARMSQEKQVTVLELFRVS
SYRQPIIISIVLQLSQQLSGINAVFYYSTGIFKDAGVQEPIYATIGAGVVNTIFTVVSLELVERAG
RRTLHMIGLGGMAFCSTLMTVSLLLKDNYNGMSFVCIGAILVFVAFFEIGPGPIPWFIVAELFSQG
PRPAAMAVAGCSNWTSNFLVGLLFPSAAHYLGAYVFIIFTGFLITFLAFTFFKVPETRGRTFEDIT
RAFEGQAHGADRSGKDGVMEMNSIEPAKETTTNV

The SCL2A nucleic acid can be, but is not limited to, a DNA, an RNA, an mRNA, a double stranded nucleic acid, a single stranded nucleic acid, a plasmid, an expression vector, a viral vector, or a CRISPR construct. One or more nucleotides or nucleobases in the nucleic acid may be modified. Viral vectors include, but are not limited to, retroviral vectors, gammaretrovirus, alphavirus, adenovirus, adeno-associated virus, vaccinia virus, herpes virus, and lentivirus. A virus vector can be engineered to transform T cells.

A T cell can be modified to overexpress a GLUT protein by introducing into the T cell a SCL2A nucleic acid. Introducing the SCL2A nucleic acid into the T cell may be done using any method available in the art. For example, the SCL2A nucleic acid can be introduced into a T cell by various transfection or transformation methods known in the art. SCL2A nucleic acid can be introduced into a T cell using a viral vector or a non-viral vector. Methods of introducing a nucleic acid into a cell include, but are not limited to, viral vectors, microinjection, microprojectile bombardment (e.g., gene gun), electroporation, lipofection, and CRISPR (e.g., CRISPR-Cas9) systems.

In some embodiments, a marker gene, such as a GFP, is introduced with the SCL2A nucleic acid into the T cell to aid in monitoring transfection. In some embodiments, the marker gene allows for selection of transfected T cells.

In some embodiments, the GLUT-overexpressing T cell is further modified. The further modification can be done prior to modifying the T cell to overexpress a GLUT gene, simultaneously with modifying the T cell to overexpress a GLUT gene, after modifying the T cell to overexpress a GLUT gene, or a combination thereof.

The further modification can add one or more desired functions to the T cell. In some embodiments, the T cell is further modified by introduction of a nucleic acid encoding an additional gene. The T cell can be further modified by introducing into the T cell, one or more nucleic acids encoding one or more of: a T cell receptor (TCR), an αβ TCR, a γδ TCR, a CAR, a first generation CAR, a second generation CAR, a third generation CAR, a fourth generation CAR, a secreted cytokine (including, but not limited to, IL-12, IL-18, IL-15, and IL-7), a cytokine receptor, a chimeric cytokine receptor, a CD40L, a4-1BBL, a dominant-negative TGF-β receptor II, a constitutively active Akt, or an antibody-like protein (including, but not limited to, a single chain variable fragment (scFv), a nanobody, or a bispecific T-cell engager (BiTE)), or combinations thereof. The T cell can be further modified by introducing in the T cell one or more of: a tumor mRNA, total tumor mRNA, slow cycling cancer cell mRNA, or cancer stem cell mRNA. In some embodiments, the T cell is modified to alter or delete one or more genes normally expressed in the T cell.

In some embodiments, the GLUT-overexpressing T cell expresses, is engineered to express, or has been engineered to express an antigen-specific receptor. The antigen-specific receptor can be, but is not limited to, a T cell receptor (TCR) or a chimeric antigen receptor (CAR). The antigen-specific receptor can be an endogenous antigen-specific receptor or a heterologous or recombinant antigen-specific receptor. An antigen-specific receptor is a receptor that specifically binds to and immunologically recognizes an antigen, or an epitope thereof, such that binding of the receptor to the antigen, or the epitope thereof, elicits an immune response, such as, but not limited to, activation of the T cell. The antigen-specific receptor can be, but is not limited to, a T cell receptor (TCR) or a CAR. An antigen-specific TCR generally comprises two polypeptides a α chain and a β chain or γ chain and a δ chain. The antigen-specific receptor can be an endogenous TCR, a heterologous TCR, or a recombinant TCR. In some embodiments, the antigen-specific receptor recognizes an antigen expressed by a tumor in a subject to be treated with the GLUT-overexpressing T cell. A CAR typically comprises an antigen binding domain of an antibody fused to a transmembrane and an intracellular signally domain such as the intracellular signally domain of a TCR (e.g., a CD3 signaling domain). In some embodiments, the TCR or CAR recognized a shared tumor antigen. In some embodiments, the CAR is modified such that it has reduced or no binding affinity for a Fc receptor.

In some embodiments, the GLUT-overexpressing T cells are expanded prior to modifying the T cells to overexpress the GLUT gene. In some embodiments, the T cells are not expanded prior to modifying the T cells to overexpress the GLUT gene. In some embodiments, the GLUT-overexpressing T cells are expanded after modifying the T cells to overexpress the GLUT gene, but prior to administration of the GLUT-overexpressing T cells to the subject. In some embodiments, the GLUT-overexpressing T cells are not expanded after modifying the T cells to overexpress the GLUT gene. The expansion of the numbers of T cells can be done using any method known in the art for expanding T cell numbers.

In some embodiments, the described GLUT-overexpressing T cells exhibit one or more of: increased survival, increased growth, increased differentiation, increased activation, increased glucose utilization, increased glucose uptake, increase fitness, increased metabolic fitness, increased tumor infiltration, or increased anti-tumor activity compared to a genetically similar T cell that does not overexpress a GLUT protein when measured under the same conditions.

In some embodiments, the described GLUT-overexpressing T cells exhibit one or more of: increased survival, increased growth, increased differentiation, increased activation, increased glucose utilization, increased glucose uptake, increase fitness, increased metabolic fitness, increased tumor infiltration, or increased anti-tumor activity in low glucose or glucose-restricted conditions compared to a genetically similar T cell that does not overexpress a GLUT gene when measured under the same conditions. In some embodiments, glucose restricted conditions have a glucose concentration of less than about 125 mg/dL, less than about 100 mg/dL, less than about 90 mg/dL, less than about 80 mg/dL, less than about 70 mg/dL, or less than about 60 mg/dL.

### C. Formulation

In some embodiments, the GLUT-overexpressing T cells are formulated with one or more additional pharmaceutically active ingredients.

In some embodiments, the described GLUT-overexpressing T cells and/or one or more additional therapeutics are combined with one or more pharmaceutically acceptable excipients to form a pharmaceutical composition. Pharmaceutically acceptable excipients (excipients) are substances other than an active pharmaceutical ingredient (API, therapeutic product) that are intentionally included with the API (molecule). Excipients do not exert or are not intended to exert a therapeutic effect at the intended dosage. Excipients may act to a) aid in processing of the API during manufacture, b) protect, support, or enhance stability, bioavailability or subject acceptability of the API, c) assist in product identification, and/or d) enhance any other attribute of the overall safety, effectiveness, of delivery of the API during storage or use. A pharmaceutically acceptable excipient may or may not be an inert substance. Excipients include, but are not limited to: adjuvants, absorption enhancers, anti-adherents, anti-foaming agents, anti-oxidants, binders, buffering agents, carriers, coating agents, colors, delivery enhancers, delivery polymers, dextran, dextrose, diluents, disintegrants, emulsifiers, extenders, fillers, flavors, glidants, humectants, lubricants, oils, polymers, preservatives, saline, salts, solvents, sugars, suspending agents, sustained release matrices, sweeteners, thickening agents, tonicity agents, vehicles, water-repelling agents, and wetting agents.

The GLUT-overexpressing T cells, or pharmaceutical composition thereof, can be formulated for administration to the subject by any suitable route. Suitable routes include, but are not limited to, parenteral administration, intravenous administration, intratumoral administration, intraarterial administration, intraperitoneal administration, and injection.

The GLUT-overexpressing T cell can be provided as a single isolated T cell or as a population of T cells. In some embodiments, a population of T cells comprises an essentially clonal population of T cells derived from a single GLUT-overexpressing T cell. In some embodiments, the population of T cells is a substantially homogenous population of GLUT-overexpressing T cells. In some embodiments, a population of T cells comprises T cells in which all or substantially all of the T cells in the population are modified to overexpress a GLUT gene. In some embodiments, the population of T cells comprises a population of T cells in which less than all of the T cells are modified to overexpress the GLUT gene. The percentage of GLUT-overexpressing T cells in a population of T cells may be at least 10%, at least 20%, at least 30%, at least 40%, at least 50%, at least 60%, at least 70%, at least 80%, at least 90%, at least 95%, or 100%. In some embodiments, the population of T cells is a heterogenous population with respect to the type of T cell.

In some embodiments, the GLUT-overexpressing T cells are provided in a population of cells that includes one or more additional cells other than T cells. The additional cell can be, but is not limited to, an antigen presenting cell, a dendritic cell, a B cell, a macrophage, a neutrophil, an erythrocyte, an endothelial cell, an epithelial cell, a parenchymal cell, or a cancer cell.

### D. Methods of Use

Described are methods of using the GLUT-overexpressing T cells to provide an immune response in a subject. The methods comprise administered the GLUT-overexpressing T cells to a subject in need of such immune response. In some embodiments, the described methods can be used to treat cancer. In some embodiments, the described methods can be used to induce an immune response against a cancer. The methods comprises administering to subject any of the T cells described herein, or a population thereof, or a composition comprising any of the T cells described herein, in an amount effective to treat or prevent the disease in the subject.

Described are methods for treatment of a cancer in a subject comprising, administering to the subject a composition comprising an effective dose of GLUT-overexpressing T cells. The methods of treating cancer include adoptive cell therapy using any of the described GLUT-overexpressing T cells or pharmaceutical compositions containing the GLUT-overexpressing T cells.

In some embodiments, methods of treating cancer using GLUT-overexpressing T cells comprise: (a) modifying one or more T cells to form GLUT-overexpressing T cells; and (b) administering the GLUT-overexpressing T cells to the subject.

In some embodiments, the methods of treating cancer using GLUT-overexpressing T cells comprise: (a) obtaining or having obtained one or more T cells from the subject or a donor subject; (b) modifying the T cells to form GLUT-overexpressing T cells; and (c) administering the GLUT-overexpressing T cells to the subject.

In some embodiments, the methods of treating cancer using GLUT-overexpressing T cells comprise: (a) obtaining or having obtained one or more T cells; (b) modifying the T cells to form GLUT-overexpressing T cells; and (c) administering the GLUT-overexpressing T cells to the subject.

In some embodiments, the methods of treating cancer using GLUT-overexpressing T cells comprise: (a) obtaining or having obtained one or more modified T cells; (b) further modifying the modified T cells to form GLUT-overexpressing T cells; and (c) administering the GLUT-overexpressing T cells to the subject.

The T cell can be any T cell as described for GLUT-overexpressing T cells, including, but not limited to, a T cell from a subject, a T cell from an allogeneic subject, or a cultured T cell. A cultured T cell can be a previously modified T cell. In some embodiments, the culture T cell is a CAR T cell. An allogenic source can be the same species or a different species. In some embodiments, the subject or donor subject is immunized with an antigen prior to obtaining the one or more T cells from the subject or donor subject. The antigen can be an antigen associated with the cancer to be treated in the subject.

Modifying the T cells to form GLUT-overexpressing T cells comprises introducing into the T cell a heterologous SCL2A nucleic acid for expression of a GLUT protein. In some embodiments, the heterologous SCL2A nucleic acid comprises a SCL2A1 coding sequence (e.g., SEQ ID NO: 1, 5, or 7) and/or SCLA3 coding sequence (e.g., SEQ ID NO: 3, 6 or 8). In some embodiments, the heterologous SCL2A nucleic acid comprises a sequence having at least 70%, at least 80%, at least 90%, at least 95%, at least 97%, at least 98%, at least 99%, or 100% to SEQ ID NO: 1, 3, 5, 6, 7, or 8. In some embodiments, the heterologous SCL2A nucleic acid encodes a protein having at least 70%, at least 80%, at least 90%, and least 95%, at least 97%, at least 98%, at least 99%, or 100% amino acid sequence identity to SEQ ID NO: 2 or 4, wherein the encoded polypeptide retains the glucose transport activity.

The heterologous SCL2A nucleic acid encoding the GLUT protein can be introduced into the T cell using any means available in the art. Such means include, but are not limited to, infecting the T cell with a virus or retrovirus containing the heterologous SCL2A nucleic acid, using a CRISPR-Cas system, or transfecting the cell with a DNA or RNA containing the heterologous SCL2A nucleic acid.

In some embodiments, the T cell is further modified. The further modification can be, but is not limited to, introduction in the T cell, one or more additional nucleic acids. In some embodiments, the one or more nucleic acids encode one or more of: a TCR, an αβ TCR, a γδ TCR, a CAR, a first generation CAR, a second generation CAR, a third generation CAR, a fourth generation CAR, a secreted cytokine, a cytokine receptor, a chimeric cytokine receptor, a CD40L, a4-1BBL, a dominant-negative TGF-β receptor II, a constitutively active Akt, or an antibody-like protein. In some embodiments, the one or more nucleic acids comprises: a tumor mRNA, total tumor mRNA, slow cycling cancer cell mRNA, or cancer stem cell mRNA. The T cell can be modified prior to, concurrent with, or after modifying the T cells to form GLUT-overexpressing T cells.

The one or more additional nucleic acids can be introduced into the T cell using any means available in the art. Such means include, but are not limited to, infecting the T cell with a virus or retrovirus containing the one or more additional nucleic acid, using a CRISPR-Cas system, or transfecting the cell with a DNA or RNA containing the one or more additional nucleic acid.

In some embodiments, the methods comprise expanding the T cells. Expanding the T cells can be done prior to or after modifying the T cells to form GLUT-overexpressing T cells. In some embodiments, the T cells are expanded prior to modifying the T cells to overexpress the GLUT protein. In some embodiments, the T cells are not expanded prior to modifying the T cells to overexpress the GLUT protein. In some embodiments, the T cells are expanded after modifying the T cells to overexpress the GLUT protein, but prior to administration of the modified T cells to the subject. In some embodiments, the T cells are not expanded after modifying the T cells to overexpress the GLUT protein. The expansion of the numbers of T cells can be done using any method known in the art for expanding T cell numbers.

In some embodiments, administering the GLUT-overexpressing T cells to the subject comprises administering to the subject a population of T cells. In some embodiments, a population of T cells comprises an essentially clonal population of T cells derived from a single GLUT-overexpressing T cell. In some embodiments, the population of T cells is a substantially homogenous population of GLUT-overexpressing T cells. In some embodiments, a population of T cells comprises T cells in which all or substantially all of the T cells in the population are modified to overexpress a GLUT gene. In some embodiments, the population of T cells comprises a population of T cells in which less than all of the T cells are modified to overexpress the GLUT gene. The percentage of GLUT-overexpressing T cells in a population of T cells may be at least 10%, at least 20%, at least 30%, at least 40%, at least 50%, at least 60%, at least 70%, at least 80%, at least 90%, at least 95%, or 100%. In some embodiments, the population of T cells is a heterogenous population with respect to the type of T cell.

In some embodiments, the GLUT-overexpressing T cells are administered to a subject in a population of cells that includes one or more additional cells other than T cells. The additional cell can be, but is not limited to, an antigen presenting cell, a dendritic cell, a B cell, a macrophage, a neutrophil, an erythrocyte, an endothelial cell, an epithelial cell, a parenchymal cell, or a cancer cell.

Treating cancer includes, but is not limited to, reducing or inhibiting cancer cell growth, reducing or inhibiting tumor growth, reducing tumor progression, reducing tumor mass, inhibiting or reducing metastasis, reducing or inhibiting the development of metastatic cancer, and/or increasing survival or prolonging life of the subject. In some embodiments, administration of GLUT-overexpressing T cells enhances T cell infiltration of the tumor.

In some embodiments, the described methods can be used to treat cancer in a human. In some embodiments, the described methods can be used to treat cancer in non-human animals or mammals. A non-human mammal can be, but is not limited to, a mouse, a rat, a rabbit, a dog, a cat, a pig, a cow, a sheep, a horse, or a non-human primate.

The term cancer includes diseases generally characterized by inappropriate cellular proliferation, or abnormal or excessive cellular proliferation. Cancers amenable to treatment using GLUT-overexpressing T cells include noninvasive, invasive, superficial, papillary, flat, metastatic, localized, unicentric, multicentric, low grade, and high grade tumors. These growths may manifest themselves as any of a lesion, a polyp, a neoplasm, a papilloma, a malignancy, a sarcoma, a carcinoma, or lump, or any other type of solid tumor.

In some embodiments, cancers amenable to treatment using the described GLUT-overexpressing T cells include cancers comprising a solid tumor and cancers having a tumor or tumor cells that demonstrate high glycolysis activity, glycolytic switch, or increase glucose uptake. Glycolytic activity of cancers can be used to provide information about the pathologic differentiation and staging of tumors. In some embodiments, the tumor or tumor cells have increased glucose uptake relative to normal non-cancerous cells adjacent to the tumor. In some embodiments, the tumor or tumor cells have increased glucose metabolism relative to normal non-cancerous cells adjacent to the tumor. In some embodiments, the tumor or tumor microenvironment as decreased glucose concentration relative to normal non-cancerous tissue adjacent to the tumor. Glucose uptake by a solid tumor can be measured using methods known in the art. Such methods include, but are not limited to, positron emission tomography (PET) with a radiolabeled analog of glucose (e.g., 18F-fluorodeoxyglucose) and magnetic resonance spectroscopy (MRS) to image the conversion of 13C-labeled pyruvate to lactate. In some embodiments, glucose uptake and metabolism by a tumor or tumor cells is determined using positron emission tomography (PET). PET imaging uses a radioisotope-labeled glucose tracer, 18F-fluorodeoxyglucose (18F-FDG), to identify areas of increase glucose uptake/metabolism in the body. The labelled-glucose analogue is transported into the cells by glucose transporters (e.g., GLUT1), and consequently phosphorylated by the hexokinase to produce 18F-FDG-6-phosphate (18F-FDG-6-p). After entering the cell, 18F-FDG-6-p is trapped and accumulates in the cytoplasm since this molecule cannot be further metabolized. Therefore, the accumulated amounts of 18F-FDG-6-p are used to identify and confirm the presence of solid tumors (showing increased glycolytic flux). Thus, the tumor amenable to treatment with the described GLUT-overexpressing T cells can be, but is not limited to, a tumor that accumulates increase 18F-FDG-6-phosphate as determined by PET scan relative to nearby non-cancerous tissue.

The cancer can be, but is not limited to, pancreas, skin, brain, cervical, liver, gall bladder, stomach, lymph node, breast, lung, head and neck, larynx, pharynx, lip, throat, heart, kidney, muscle, colon, prostate, thymus, testis, uterine, ovary, cutaneous, and subcutaneous cancers. Skin cancer can be, but is not limited to, melanoma and basal cell carcinoma. Breast cancer can be, but is not limited to, ER positive breast cancer, ER negative breast cancer, and triple negative breast cancer.

In pharmacology, a route of administration is the path by which a drug, fluid, or other substance is brought into contact with the body. In general, methods of administering drugs and nucleic acids for treatment of a mammal are well known in the art and can be applied to administration of the described GLUT-overexpressing T cells. The described T cells can be administered via any suitable route in a preparation appropriately tailored to that route. In general, any suitable method recognized in the art for delivering a T cell or ACT therapeutic can be adapted for use with a herein described compounds and compositions. Routes of administration include, but are not limited to, parenteral, local, direct injection, intraparenchymal, intratumoral, intramuscular, systemic, intravascular, intravenous, intraarterial, intraventricular, intralymphatic, intraperitoneal, or intracranial administration.

### E. Additional Therapies

The methods can be combined with other cancer therapeutics or active pharmaceutical ingredients, including, but not limited to, immune checkpoint therapy, surgical resection, chemotherapy, and radiation therapy.

In some embodiments, GLUT-overexpressing T cells are combined with one or more additional therapeutics. The GLUT-overexpressing T cells can be administered to a subject prior to administration of the one or more additional therapeutics, concurrently with the one or more additional therapeutics, or subsequent to the one or more additional therapeutics.

Immune checkpoint therapy can be, but is not limited to, administration of one or more immune checkpoint inhibitors. Immune checkpoint molecules refer to a group of immune cell surface receptor/ligands which induce T cell dysfunction or apoptosis. These immune checkpoint molecules attenuate excessive immune reactions and ensure self-tolerance. Tumor cells harness the suppressive effects of these checkpoint molecules. Immune checkpoint molecules include, but are not limited to, Cytotoxic T Lymphocyte Antigen-4 (CTLA-4), Programmed Death 1 (PD-1), Programmed Death Ligand 1 (PD-L1), Lymphocyte Activation Gene-3 (LAG-3), T cell Immunoglobulin Mucin-3 (TIM3), Killer Cell Immunoglobulin-like Receptor (MR), B- and T-Lymphocyte Attenuator (BTLA), Adenosine A2a Receptor (A2aR), and Herpes Virus Entry Mediator (HVEM). Immune checkpoint inhibitors include molecules that prevent immune suppression by blocking the effects of immune checkpoint molecules. Immune checkpoint inhibitors include, but are not limited to, antibodies and antibody fragments, nanobodies, diabodies, soluble binding partners of checkpoint molecules, small molecule therapeutics, and peptide antagonists. An immune checkpoint inhibitor can be, but is not limited to, a PD-1 and/or PD-L1 antagonist. A PD-1 and/or PD-L1 antagonist can be, but is not limited to, an anti-PD-1 or anti-PD-L1 antibody. Anti-PD-1/PD-L1 antibodies include, but are not limited to, nivolumab, pembrolizumab, pidilizumab, and atezolizumab.

It is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

### EXAMPLES

### Example 1. Increased Glucose Consumption by Cancer Cells Inhibits Immune Response

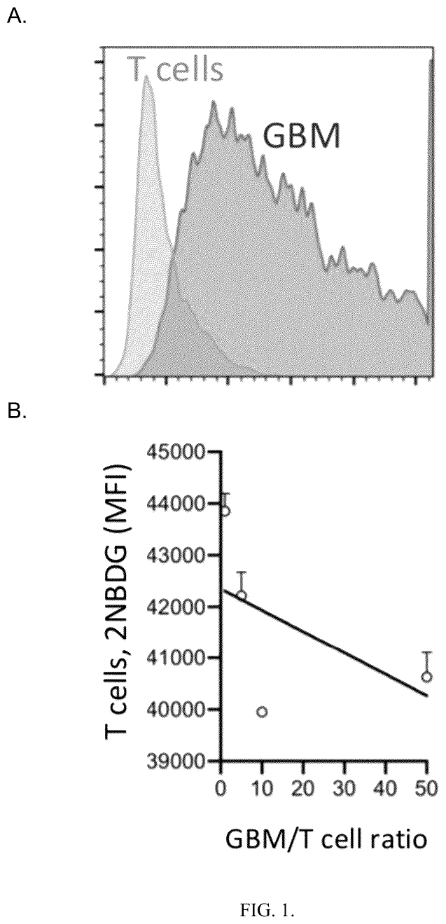

dsRed labeled T cells were co-cultured with HGG cells in the presence of the fluorescent glucose analog 2NBDG (5 μg/mL). After 24 h, the amount of glucose uptake was compared using flow cytometry. HGG cells incorporated significantly more glucose than T cells. As shown in FIG. 1A, in GBM cells out compete T cells for glucose uptake.

2NBDG incorporation in T cells was also measured in the presence of increased amount of tumor cells. The results show a negative correlation between T cell glucose uptake and the number of tumor cells. As shown in FIG. 1B, and the ratio of GBM cells to T cells increases, the uptake of glucose by T cells decreases for the same number of T cells.

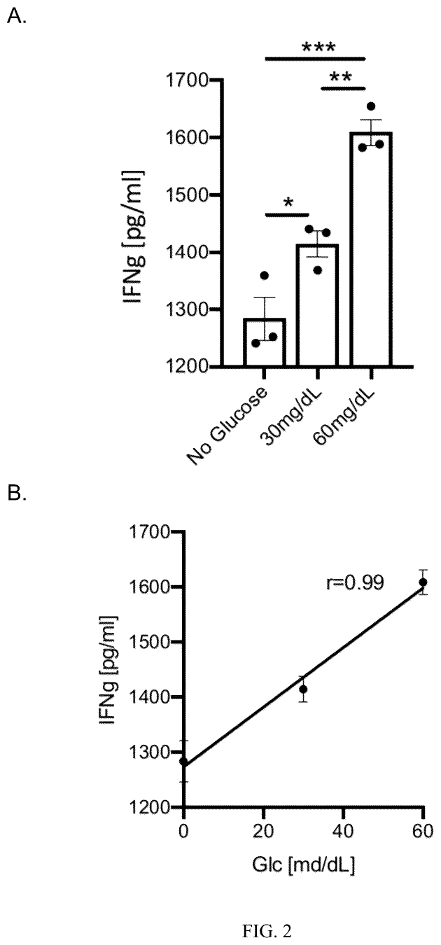

IFNγ was measured in the supernatant of T cell cultures containing different concentrations of glucose. The results demonstrate a positive correlation between glucose concentration and level of IFNγ secreted by T cells. Further, as glucose becomes limiting the ability of T cells to be activated decreases, as evidenced by decreased IFNγ production (FIG. 2).

Together, this data functionally illustrates the critical role of glucose for T cells activation and that tumor cells, competing for glucose, restrict the availability of the nutrient resulting in decreased T cell glucose uptake and activation.

Cancer cells, such as glioma cells, impose glucose restriction thereby contributing to T cell hyporesponsiveness. Tumor cells create therefore a specific metabolic environment that can contribute to tumor immune evasion. Overexpression of glucose transporters in T cells can be used to stimulate glycolysis in T cells and enhance anti-tumor immune response. By increasing metabolic fitness of T cells, through overexpression of glucose transporters in the T cells, the anti-tumor response mediated by the T cells is increased.

### Example 2. Overexpression of Glucose Transporters in T Cells does not Adversely Affect T Cell Viability, Differentiation or Activation, and Improves Metabolic Fitness

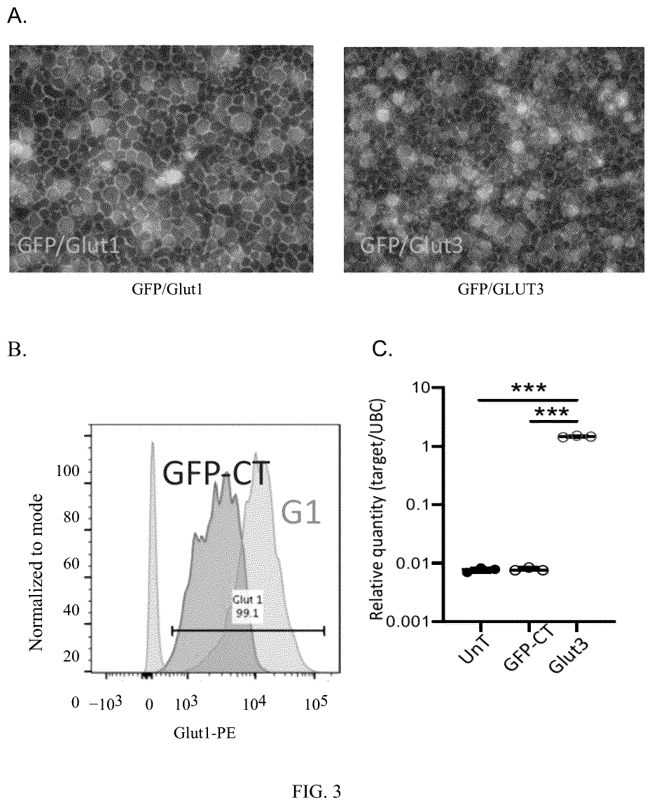

Expression vectors were made that expressed GFP and GLUT1 or GFP and GLUT3. The vectors contained nucleic acid sequences encoding GFP and either GLUT1 or GLUT3. The GFO and GLUT genes were expressed from a common promoter using a 2A element of produce both separate GPF and the GLUT proteins. After transfection of T cells, expression of GFP (as a surrogate for GLUT1 or GLUT3) was analyzed using fluorescent microscopy. Transduction efficiency was determined by GFP-positivity measured by fluorescent microscopy. As shown in FIG. 3A, both T cells expressing GFP+GLUT1 and GFP+GLUT3 expressed the GFP proteins. Overexpression of the GLUT targets was confirmed by flow cytometry and RT-qPCR. As shown in FIG. 3B, FACS sorting demonstrated that T cells transfected with expression vector encoding GFP and GLUT1 expressed both GFP and GLUT1. As shown in FIG. 3C, RT-qPCR also demonstrated in increase in expression of GLUR3 in the modified T cells.

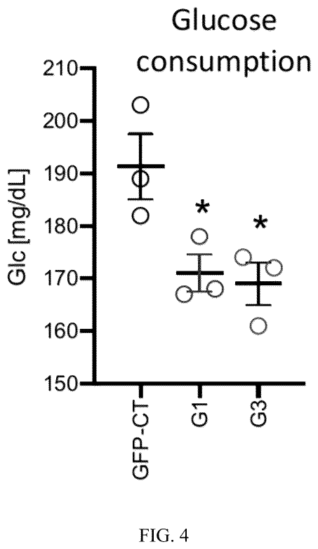

After confirmation of expression of GLUT1 and GLUT3 in the modified T cells, the GLUT-overexpressing T cells were analyzed for glucose consumption, viability, differentiation, and activation. Control and GLUT-overexpressing T cells were incubated in growth media containing glucose. Glucose concentration was compared in the media after 24 h culture of the different T cell populations. As shown in FIG. 4, T cells overexpressing GLUT1 or GLUT3 exhibited higher glucose consumption than control T cells transfected with GFP alone as evidence by the decreased levels of glucose remaining in the media.

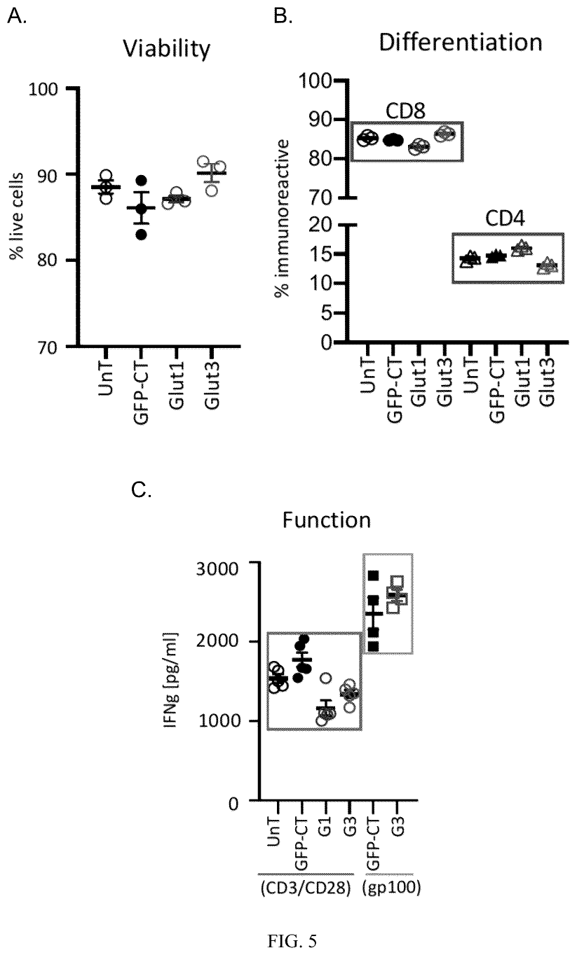

T cell viability was measured by flow cytometry using live/dead dye assay. Overexpression of GLUT1 or GLUT3 in T cells did not substantially affected viability, differentiation, activation of the T cells grown in optimum conditions (unrestricted glucose). As shown in FIG. 5A, percent live cells as not adversely affected by overexpression of the GLUT genes in the T cells.

Percent of CD8+ and CD4+ cells were compared by flow cytometry. Similarly, differentiation was not affected in the modified T cells (FIG. 5B). The percent immunoreactive CD8+ and CD4+ was not adversely affected by overexpression of GLUT1 or GLUT3 in T cells compared to untransfected control T cells or T cells transfected with GFP alone.

IFNγ production in response to 24 h treatment with anti-CD3 and CD28 antibodies was measured using ELISA. GLUT-overexpressing T cells were equally activated by contact with either CD2/CD38 expressing cells or gb100 expressing cells as determined by IFNγ production. CD3/CD28 expressing cells activate the T cells in an antigen-independent manner, while gp100 expressing cells activate the T cells in an antigen-dependent manner. Thus, both antigen-independent and antigen-dependent activation were unaffected by overexpression of GLUT1.

Overexpression of GLUT1 or GLUT3 increased murine T cell glucose consumption and did not negatively impact their viability, differentiation and activation in optimal nutrient-replete conditions.

### Example 3. GLUT Overexpression in T Cells Provides a Survival Advantage in Glucose-Restricted Conditions

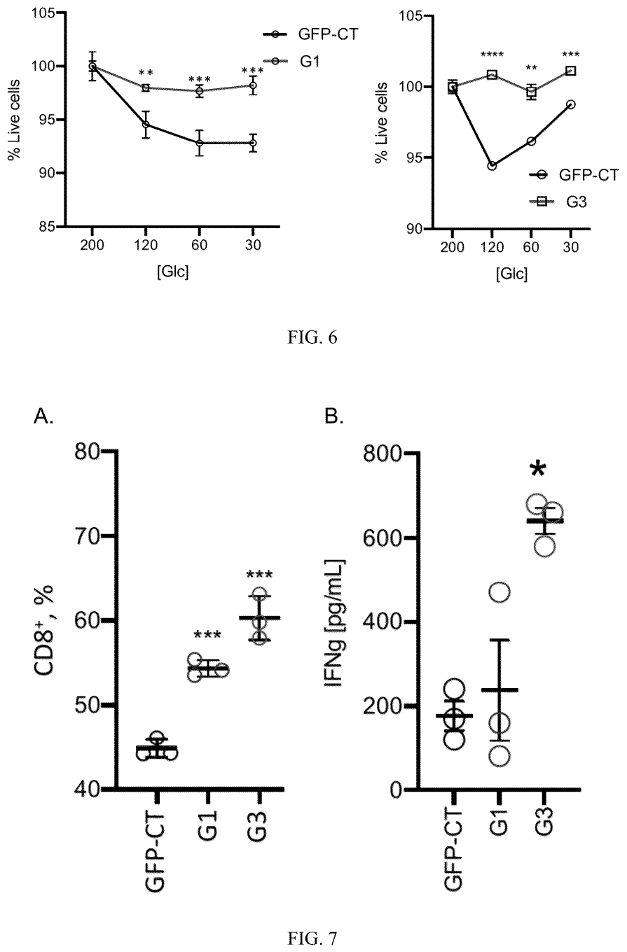

T cells were grown in media containing 200 mg/dL glucose and transfected with expressing vectors encoding GLUT1 or GLUT3 on day 2. On day 3, the T cells were transferred to media containing 30 mg/dL, 60 mg/dL, 120 mg/dL, or 200 mg/dL glucose. The percentage of live T cells, measured by flow cytometry using live/dead dye, under different concentrations of glucose was then determined. As shown in FIG. 6, the present live cells was increased in GLUT1-overexpressing T cells or GLUT3-overexpressing T cells compared to control T cell expressing GFP. Thus, overexpression of GLUT genes confers a survival advantage to the T cells in low glucose or glucose-restricted conditions.

### Example 4. GLUT Overexpression in T Cells Improved Differentiation and Activation in Glucose-Restricted Conditions

T cells were isolated from mice and incubated with Kluc RNA transfected dendritic cells on starting on day 0 and grown in the presence of 200 mg/dL glucose. The dendritic cells were electroporated with RNA from a Kluc tumor cell line. On day 2, the T cells were transfected with virus containing nucleic acid encoding GLUT1 or GLUT3. On day 7, the T cells transferred media containing 60 mg/mL glucose (glucose restricted conditions) and co-cultured with tumor cells. On day 9, T cell differentiation, as determined by percent CD8+ cells using flow cytometry, and activation, as determined by IFNγ secretion using ELISA were quantified. As shown in FIG. 7A, T cells overexpressing GLUT1 or GLUT3 exhibited higher levels of differentiation to form CD8+ cytotoxic T cells compared to control T cells expressing GFP. As shown in FIG. 7B, GLUT3-overexpressing T cells were also more highly activated compared to control T cells expressing GFP, as determined by IFNγ production. GLUT1 or GLUT3 overexpression improved cytotoxic CD8+ T cell differentiation, activation, and tumor targeting in glucose-restricted conditions.

### Example 5. GLUT Overexpression in Human T Cells Increased Glucose Utilization, Survival and Expansion in Glucose-Restriction Conditions

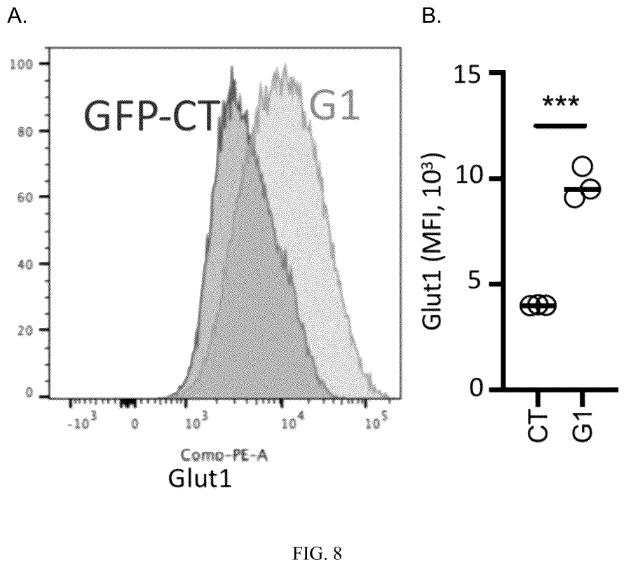

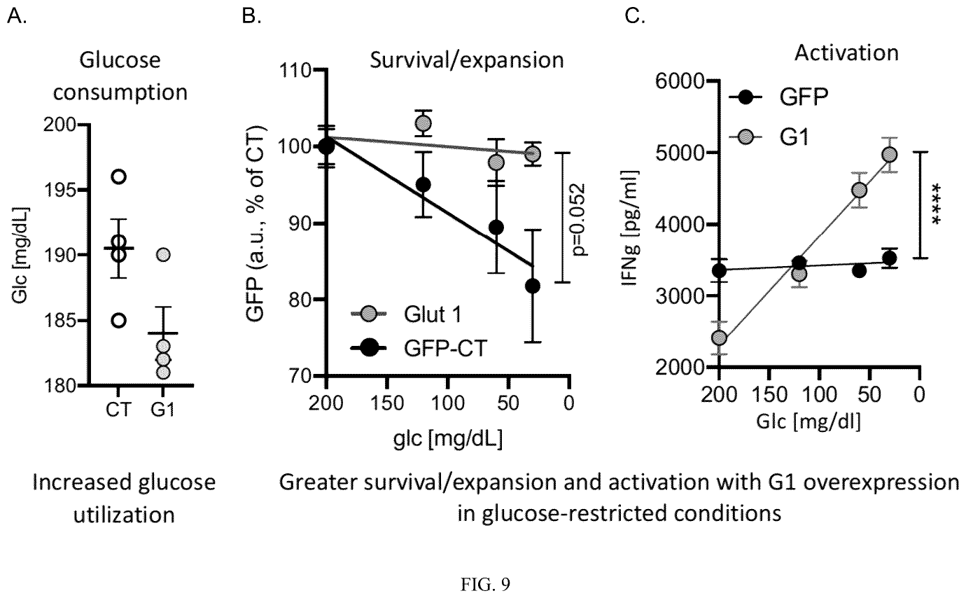

Human T cells were transfected with viral vectors containing nucleic acid encoding GFP and either GLUT1. As shown in FIG. 8, the modified T cells overexpress GLUT1. As shown in FIG. 9, GLUT1-overexpressing T cells exhibited increased glucose consumption (FIG. 9A, decreased glucose in the media correlated with increased glucose consumption by the T cells), increased survival (FIG. 9B), and increased activation (FIG. 9C). Survival of T cells after 24 h of culture in the different glucose concentrations was quantified by measuring GFP using a Cytation 3 cell imaging multi-mode reader (Bio Tek). The number of GFP positive cells was used as a surrogate for T cell survival. IFNγ production was used to determine activation level. T cells were activated using anti CD3/CD28 antibodies in the different glucose concentrations. IFNγ production was measured using ELISA 24 h after stimulation.

As shown in FIGS. 9B and 9C, survival and activation were both increased in GLUT1-overexpressing T cells in glucose-restricted conditions. The survival and differentiation advantage increase in decreasing availability of glucose.

Overexpression of GLUT1 or GLUT3 increases glucose consumption of human T cells, provides a survival advantage, and enhances activation and human T cell anti-tumor targeting in glucose-restricted conditions.

### Example 6. GLUT Overexpression in T Cells Increase Intratumoral Trafficking In Vivo

Human glioma cells (U87) tumor cells were implanted in mice on day 0. The U87 tumor cells express iRFP720, which form a visible tumor imaged using IVIS in vivo imager. On day 28, control T cells or GLUT-overexpressing T cells, also expressing luciferase and/or GFP, were administered to the mice. Tumor volumes were quantified using in vivo imager. Intratumoral T cell trafficking was compared based on luciferase activity measured using in vivo imager. 48 h after T cell injection, GFP⁺ T cells were observed by flow cytometry.

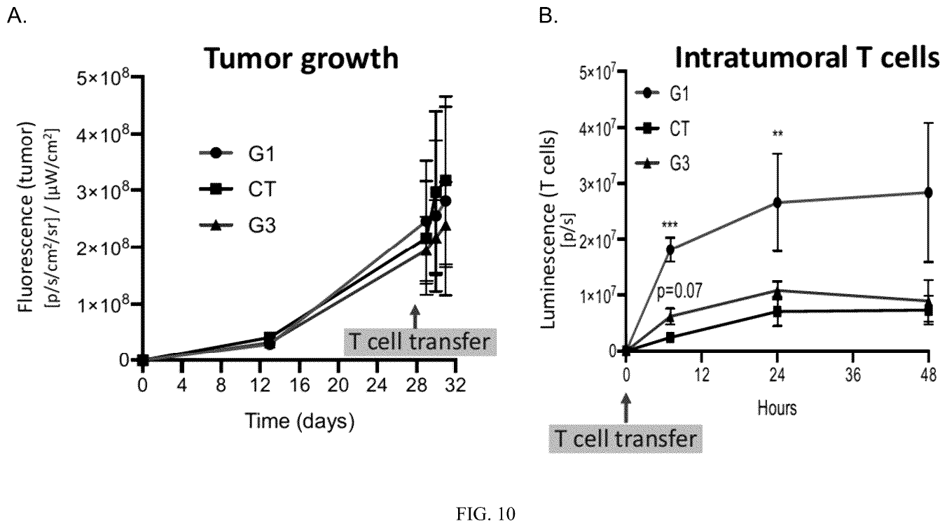

While the T cells overexpressed GLUT, the T cells were not specific for the U87 tumor. Thus, the T cells were not targeted to the tumor and were not expected to be efficacious in inducing an immune response against the tumor. FIG. 10A illustrates that tumors were relatively similar in size between the different mice. While the T cells were not expected to be efficacious in inducing an immune response against the tumor, GLUT-overexpressing T cells were nevertheless found to infiltrate the tumor at a higher rate than control T cells not overexpressing GLUT (FIG. 10B).

48 h after T cell injection, fluorescent microscopy of GFP was used to observe TILs. GLUT-overexpressing T cells were TILs were observed in the tumors at a higher rate that control T cells that did not overexpress GLUT. The increased infiltration rate is likely because of the higher fitness of the GLUT-overexpressing T cells in the glucose-restricted microenvironment of the tumor. Thus, not only did overexpression of GLUT in T cells not adversely affect tumor infiltration, tumor infiltration was increased even in the absence of tumor specificity (absence of targeting).

GLUT1 or GLUT3 overexpression improved human naïve T cell tumor infiltration.

### Example 7. Overexpression of GLUT Genes in CAR-T Cells

In example 6, behavior of GLUT-overexpressing T cells that were not specific for the tumor was analyzed in vivo. In this experiment, GLUT-overexpressing T cells further contained a tumor specific CAR, CD70 CAR.

To overcome the inhibitory effect of the tumor microenvironment involving glucose restriction, we engineered CD70 CAR T cells to overexpress glucose transporters (GLUT1 or GLUT3), i.e., GLUT-overexpression CD70 CAR T cells. Efficacy of GLUT-overexpressing CD70 CAR T cells in treating a tumor was then analyzed.

T cells were obtained that expressed the CD70 CAR. The CD70 CAR contains a CD27 CD70-binding domain (CD27 is the CD70 ligand), a transmembrane domain, a 4-1BB intracellular signaling domain and a CD3 signaling domain. CD70 CAR is triggered by binding to its target, CD70, expressed at the surface of tumor cells, resulting in tumor antigen specific T cell activation. The CD70 CAR is able to activate the T cell in the absence of a second signal. The CD70 CAR T cells were then modified to overexpress GLUT1 or GLUT3.

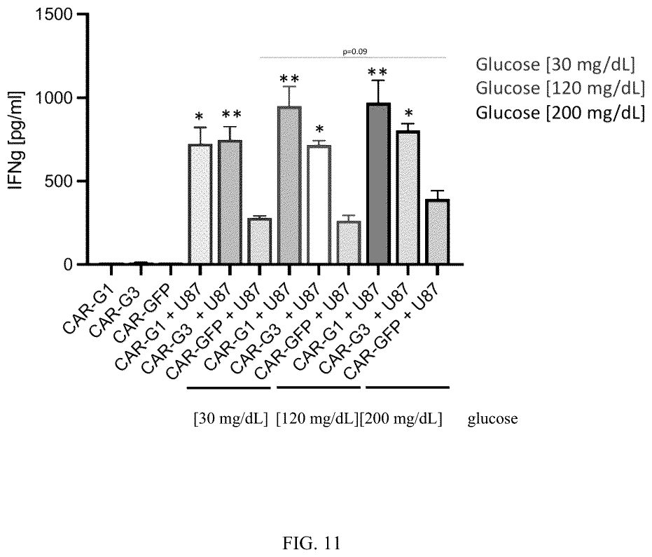

The GLUT-overexpressing CD70 CAR T cells were co-cultured with U87 (which express CD70) tumor cells at a ratio of 1:1 in varying concentrations of glucose reflecting the tumor microenvironment. After 24 h, the level of T cell activation was compared by measuring, using ELISA, the concentration of IFNγ produced compared to control GFP-expressing CD70 CAR T cells. As shown in FIG. 11, overexpression of GLUT1 or GLUT3 in the CD70 CAR T cells enhanced the activation of CD70 CAR-T cells even in glucose-restricted conditions. Overexpression of GLUT1 or GLUT3 enhanced the activation of CD70 CAR-T cells in normal and restricted glucose conditions.

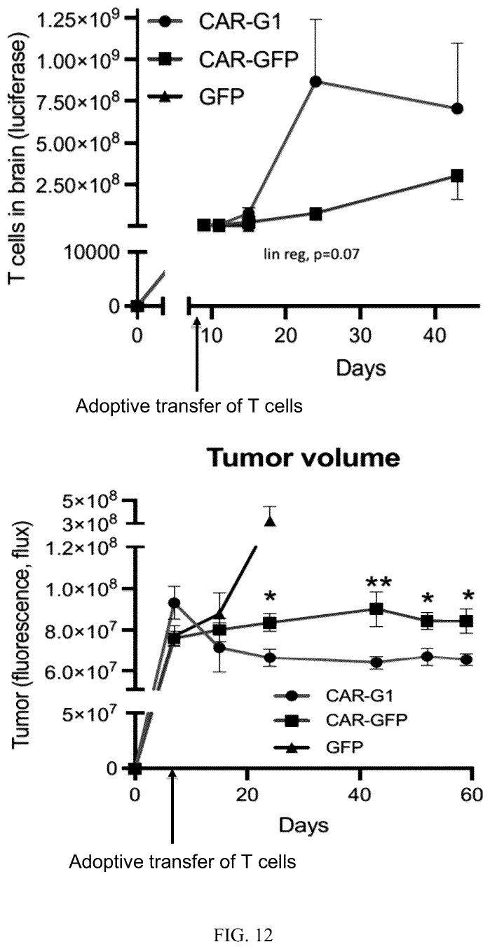

Infra-tumoral trafficking of GLUT-overexpressing CD70 CAR T cells. Transduced Luciferase expressing CD70 CAR T cells were injected i.v. nine days after intracranial implantation of human glioma cells (U87) expressing iRFP720, which form a visible tumor imaged using IVIS in vivo imager. Tumor volumes were quantified using in vivo imager. Intratumoral T cell trafficking was compared based on luciferase activity measured using in vivo imager. As shown in FIG. 12, when injected into mice having a GBM tumor, T cells expressing CD70 CAR and GLUT1 (CAR-G1) exhibited increased localization to the cancer in the brain (Top panel). As expected, CD70 CART cells (CAR-GFP) exhibited increased anti-tumor activity compared to T cells expressing only GFP. However, anti-tumor activity was further increased when the T cells also overexpressed GLUT1 (CAR-G1, lower panel). Thus, overexpression of GLUT1 in T cells improved intratumoral trafficking of CD70 CAR-T cells, resulting in reduced tumor growth.

Overexpression of GLUT1 improved intratumoral trafficking of CD70 CAR-T cells, resulting in reduced tumor growth.

While the examples illustrate the utility of increasing metabolic fitness of T cells for treating glioblastoma multiforme, the described GLUT-overexpressing T cells are suitable for use in treatment of any solid tumor that has, or has the potential to create, a glucose-restricted microenvironment.

1. A GLUT-overexpressing T cell comprising: a T cell expressing one or more heterologous SCL2A nucleic acids, wherein the GLUT-overexpressing T cell overexpresses one or more glucose transporters.

2. The GLUT-overexpressing T cell of claim 1, wherein the one or more glucose transporters are selected from the group consisting of: GLUT1, GLUT 2, GLUT3, GLUT4, GLUT5 GLUT6, GLUT7, GLUT8, GLUT9, GLUT10, GLUT11, GLUT12, GLUT13, and GLUT 14.

3. The GLUT-overexpressing T cell of claim 2, wherein the one or more glucose transporters comprises GLUT1 and/or GLUT3.

4. The GLUT-overexpressing T cell of claim 1, wherein the one or more heterologous SCL2A nucleic acids is introduced into the T cell by a DNA vector, an RNA vector, a virus, a retrovirus, or a CRISPR-Cas system.

5. The GLUT-overexpressing T cell of claim 1, wherein the one or more heterologous glucose transporters is transiently overexpressed in the T cell by an mRNA introduced into the T cell.

6. A GLUT-overexpressing T cell, wherein GLUT overexpression is induced by a hormone or expression of a heterologous insulin receptor, a CD28, an IL2-R, an IL7-R, or an IL3-R in the T cell.

7. The GLUT-overexpressing T cell of claim 6, wherein the hormone is selected from the group consisting of: an insulin, a testosterone, a glucocorticoid, and a retinoic acid.

8. (canceled)

9. The GLUT-overexpressing T cell of claim 1, wherein the T cell is a primary T cell, a culture T cell, a autologous T cell, an allogeneic T cell, a T cell obtained from bone marrow, a T cell obtained from a lymph node, a T cell obtained from a thymus, a tumor infiltrating lymphocyte, a T cell obtained from a spleen, a T cell from umbilical cord blood, a universal allogenic T cell, a universal CAR T cell, a CAR T cell, a naïve T cell, an effector T cell, an effector memory T cell, a CD4+/CD8+ T cell, a helper T cell, a CD4+ T cell, a CD4+ helper T cell, a Th1 T cell, a Th2 T cell, a cytotoxic T cell, a CD8+ T cell, peripheral blood mononuclear cell (PBMC), a peripheral blood leukocyte (PBL), a memory T cell, a central memory T cell, a regulatory T cell, an αβ T cell, a γδ T cell, a modified T cell, a T cell for use in adoptive cell transfer therapy, a TCR-engineered T cell, a chimeric antigen receptor (CAR) T cell, a first generation CAR T cell, a second generation CAR T cell, a third generation CAR T cell, a fourth generation CAR T cell, dual-antigen receptor CAR T cell, or a CAR T cell having an inducible suicide gene, or a combination thereof.

10. (canceled)

11. (canceled)

12. (canceled)

13. (canceled)

14. A method of treating cancer in a subject comprising administering to the subject a pharmaceutically effective dose of the GLUT-overexpressing T cells of claim 1.

15. The method of claim 14, wherein the cancer is glioma or glioblastoma multiforme.

16. The GLUT-overexpressing T cell of claim 1, wherein the GLUT-overexpressing T cell has enhanced metabolic fitness relative to a similar T cell that does not overexpress the one or more glucose transporters.

17. (canceled)

18. A method of treating cancer in a subject comprising:
(a) obtaining a T cell from the subject or obtaining a donor T cell;
(b) modifying the T cell to overexpress one or more glucose transporters thereby forming a GLUT-overexpressing T cell;
(c) administering the GLUT-overexpressing T cell to the subject.

19. The method of claim 18, wherein modifying the T cell to overexpress the one or more glucose transporters comprises:
(a) expressing one or more heterologous SCL2A nucleic acids in the T cell, thereby expression one or more heterologous glucose transporters in the T cell;
(b) contacting the T cell with a hormone selected from the group consisting of: an insulin, a testosterone, a glucocorticoid, and a retinoic acid, thereby increasing expression of one or more endogenous glucose transporters in the T cell; or
(c) expressing a heterologous nucleic acid encoding an insulin receptor, a CD28, an IL2-R, an IL7-R, or an IL3-R in the T cell, thereby increasing expression of one or more endogenous glucose transporters in the T cell or increasing translocation of one or more endogenous glucose transporters to the T cell plasma membrane.

20. The method of claim 19, wherein expressing one or more heterologous SCL2A nucleic acids in the T cell comprises introducing into the T cell one or more nucleic acids encoding one or more SCL2A gene coding sequences.

21. The method of claim 18, wherein the one or more glucose transporters are selected from the group consisting of GLUT1, GLUT 2, GLUT3, GLUT4, GLUT6, GLUT7, GLUT8, GLUT9, GLUT10, GLUT11, and GLUT12.

22. The method of claim 20, wherein the one or more glucose transporters comprises GLUT1 and/or GLUT3.

23. (canceled)

24. (canceled)

25. The method of claim 18, further comprising modifying the T cell to express to one or more additional heterologous genes selected from the group consisting of: a T cell receptor (TCR), an αβ TCR, a γδ TCR, a CAR, a first generation CAR, a second generation CAR, a third generation CAR, a fourth generation CAR, a secreted cytokine, a cytokine receptor, a chimeric cytokine receptor, a CD40L, a4-1BBL, a dominant-negative TGF-β receptor II, a constitutively active Akt, an antibody-like protein, a nanobody, a bispecific T-cell engager, a tumor mRNA, total tumor mRNA, slow cycling cancer cell mRNA, or cancer stem cell mRNA.

26. (canceled)

27. (canceled)

28. (canceled)

29. The method of claim 18, wherein the T cell of step (a) is selected from the group consisting of: a primary T cell, a culture T cell, a autologous T cell, an allogeneic T cell, a T cell obtained from bone marrow, a T cell obtained from a lymph node, a T cell obtained from a thymus, a tumor infiltrating lymphocyte, a T cell obtained from a spleen, a T cell from umbilical cord blood, a universal allogenic T cell, a universal CAR T cell, a CAR T cell, a naïve T cell, an effector T cell, an effector memory T cell, a CD4+/CD8+ T cell, a helper T cell, a CD4+ T cell, a CD4+ helper T cell, a Th1 T cell, a Th2 T cell, a cytotoxic T cell, a CD8+ T cell, peripheral blood mononuclear cell (PBMC), a peripheral blood leukocyte (PBL), a memory T cell, a central memory T cell, a regulatory T cell, an αβ T cell, a γδ T cell, a modified T cell, a T cell for use in adoptive cell transfer therapy, a TCR-engineered T cell, a chimeric antigen receptor (CAR) T cell, a first generation CAR T cell, a second generation CAR T cell, a third generation CAR T cell, a fourth generation CAR T cell, dual-antigen receptor CAR T cell, or a CAR T cell having an inducible suicide gene, or a combination thereof.

30. The GLUT-overexpressing T cell of claim 1, wherein the GLUT-overexpressing T cell has increased survival and growth in a glucose restricted environment relative to a T cell that does not overexpress the GLUT protein.

31. The method of claim 14, wherein the method comprises adoptive cell transfer therapy.


SEQUENCE LISTING (8 sequences)

SEQ ID NO: 1 - DNA, 1476 bp, Homo sapiens
atggagcccagcagcaagaagctgacgggtcgcctcatgctggccgtgggaggagcagtgcttggctccctgcagtttggctacaacactggagtcatcaatgccccccagaaggtgatcgaggagttctacaaccagacatgggtccaccgctatggggagagcatcctgcccaccacgctcaccacgctctggtccctctcagtggccatcttttctgttgggggcatgattggctccttctctgtgggccttttcgttaaccgctttggccggcggaattcaatgctgatgatgaacctgctggccttcgtgtccgccgtgctcatgggcttctcgaaactgggcaagtcctttgagatgctgatcctgggccgcttcatcatcggtgtgtactgcggcctgaccacaggcttcgtgcccatgtatgtgggtgaagtgtcacccacagcccttcgtggggccctgggcaccctgcaccagctgggcatcgtcgtcggcatcctcatcgcccaggtgttcggcctggactccatcatgggcaacaaggacctgtggcccctgctgctgagcatcatcttcatcccggccctgctgcagtgcatcgtgctgcccttctgccccgagagtccccgcttcctgctcatcaaccgcaacgaggagaaccgggccaagagtgtgctaaagaagctgcgcgggacagctgacgtgacccatgacctgcaggagatgaaggaagagagtcggcagatgatgcgggagaagaaggtcaccatcctggagctgttccgctcccccgcctaccgccagcccatcctcatcgctgtggtgctgcagctgtcccagcagctgtctggcatcaacgctgtcttctattactccacgagcatcttcgagaaggcgggggtgcagcagcctgtgtatgccaccattggctccggtatcgtcaacacggccttcactgtcgtgtcgctgtttgtggtggagcgagcaggccggcggaccctgcacctcataggcctcgctggcatggcgggttgtgccatactcatgaccatcgcgctagcactgctggagcagctaccctggatgtcctatctgagcatcgtggccatctttggctttgtggccttctttgaagtgggtcctggccccatcccatggttcatcgtggctgaactcttcagccagggtccacgtccagctgccattgccgttgcaggcttctccaactggacctcaaatttcattgtgggcatgtgcttccagtatgtggagcaactgtgtggtccctacgtcttcatcatcttcactgtgctcctggttctgttcttcatcttcacctacttcaaagttcctgagactaaaggccggaccttcgatgagatcgcttccggcttccggcaggggggagccagccaaagtgacaagacacccgaggagctgttccatcccctgggggctgattcccaagtg

SEQ ID NO: 2 - protein, 492 aa, Homo sapiens
MEPSSKKLTGRLMLAVGGAVLGSLQFGYNTGVINAPQKVIEEFYNQTWVHRYGESILPTTLTTLWSLSVAIFSVGGMIGSFSVGLFVNRFGRRNSMLMMNLLAFVSAVLMGFSKLGKSFEMLILGRFIIGVYCGLTTGFVPMYVGEVSPTALRGALGTLHQLGIVVGILIAQVFGLDSIMGNKDLWPLLLSIIFIPALLQCIVLPFCPESPRFLLINRNEENRAKSVLKKLRGTADVTHDLQEMKEESRQMMREKKVTILELFRSPAYRQPILIAVVLQLSQQLSGINAVFYYSTSIFEKAGVQQPVYATIGSGIVNTAFTVVSLFVVERAGRRTLHLIGLAGMAGCAILMTIALALLEQLPWMSYLSIVAIFGFVAFFEVGPGPIPWFIVAELFSQGPRPAAIAVAGFSNWTSNFIVGMCFQYVEQLCGPYVFIIFTVLLVLFFIFTYFKVPETKGRTFDEIASGFRQGGASQSDKTPEELFHPLGADSQV

SEQ ID NO: 3 - DNA, 1488 bp, Homo sapiens
atggggacacagaaggtcaccccagctctgatatttgccatcacagttgctacaatcggctctttccaatttggctacaacactggggtcatcaatgctcctgagaagatcataaaggaatttatcaataaaactttgacggacaagggaaatgccccaccctctgaggtgctgctcacgtctctctggtccttgtctgtggccatattttccgtcgggggtatgatcggctccttttccgtcggactcttcgtcaaccgctttggcaggcgcaattcaatgctgattgtcaacctgttggctgtcactggtggctgctttatgggactgtgtaaagtagctaagtcggttgaaatgctgatcctgggtcgcttggttattggcctcttctgcggactctgcacaggttttgtgcccatgtacattggagagatctcgcctactgccctgcggggtgcctttggcactctcaaccagctgggcatcgttgttggaattctggtggcccagatctttggtctggaattcatccttgggtctgaagagctatggccgctgctactgggttttaccatccttcctgctatcctacaaagtgcagcccttccattttgccctgaaagtcccagatttttgctcattaacagaaaagaagaggagaatgctaagcagatcctccagcggttgtggggcacccaggatgtatcccaagacatccaggagatgaaagatgagagtgcaaggatgtcacaagaaaagcaagtcaccgtgctagagctctttagagtgtccagctaccgacagcccatcatcatttccattgtgctccagctctctcagcagctctctgggatcaatgctgtgttctattactcaacaggaatcttcaaggatgcaggtgttcaagagcccatctatgccaccatcggcgcgggtgtggttaatactatcttcactgtagtttctctatttctggtggaaagggcaggaagaaggactctgcatatgataggccttggagggatggctttttgttccacgctcatgactgtttctttgttattaaaggataactataatgggatgagctttgtctgtattggggctatcttggtctttgtagccttctttgaaattggaccaggccccattccctggtttattgtggccgaactcttcagccagggcccccgcccagctgcgatggcagtggccggctgctccaactggacctccaacttcctagtcggattgctcttcccctccgctgctcactatttaggagcctacgtttttattatcttcaccggcttcctcattaccttcttggcttttaccttcttcaaagtccctgagacccgtggcaggacttttgaggatatcacacgggcctttgaagggcaggcacacggtgcagatagatctggaaaggacggcgtcatggagatgaacagcatcgagcctgctaaggagaccaccaccaatgtc

SEQ ID NO: 4 - protein, 496 aa, Homo sapiens
MGTQKVTPALIFAITVATIGSFQFGYNTGVINAPEKIIKEFINKTLTDKGNAPPSEVLLTSLWSLSVAIFSVGGMIGSFSVGLFVNRFGRRNSMLIVNLLAVTGGCFMGLCKVAKSVEMLILGRLVIGLFCGLCTGFVPMYIGEISPTALRGAFGTLNQLGIVVGILVAQIFGLEFILGSEELWPLLLGFTILPAILQSAALPFCPESPRFLLINRKEEENAKQILQRLWGTQDVSQDIQEMKDESARMSQEKQVTVLELFRVSSYRQPIIISIVLQLSQQLSGINAVFYYSTGIFKDAGVQEPIYATIGAGVVNTIFTVVSLFLVERAGRRTLHMIGLGGMAFCSTLMTVSLLLKDNYNGMSFVCIGAILVFVAFFEIGPGPIPWFIVAELFSQGPRPAAMAVAGCSNWTSNFLVGLLFPSAAHYLGAYVFIIFTGFLITFLAFTFFKVPETRGRTFEDITRAFEGQAHGADRSGKDGVMEMNSIEPAKETTTNV

SEQ ID NO: 5 - DNA, 1479 bp, Homo sapiens
atggagcccagcagcaagaagctgaccggcagactgatgctggctgttggcggagctgttctgggctctctgcagttcggctacaacaccggcgtgatcaacgcccctcagaaagtgatcgaggaattctacaaccagacctgggtgcacagatacggcgagagcatcctgcctaccacactgacaacactgtggtccctgagcgtggccatcttttctgtcggcggcatgatcggcagcttcagcgtgggcctgttcgtgaacagattcggcagacggaactccatgctgatgatgaacctgctggccttcgtgtccgccgtgctgatgggcttttctaagctgggcaagagcttcgagatgctgatcctgggaagattcatcatcggcgtgtactgcggcctgaccaccggctttgtgcctatgtacgtgggcgaagtgtcccctacagctctgagaggtgctctgggaacactgcaccagctgggcatcgttgtgggaatcctgatcgcccaggtgttcggcctggatagcatcatgggcaacaaggacctgtggcctctgctgctgagcatcatcttcatccccgctctgctgcagtgcatcgtgctgcctttctgtcctgagagccccagattcctgctgatcaaccggaacgaggaaaaccgggccaagagcgtgctgaagaaactgagaggcaccgccgacgtgacccacgacctgcaagagatgaaggaagagtccagacagatgatgcgcgagaagaaagtgaccatcctggaactgttcagaagccccgcctacagacagcccatcctgattgctgtggtgctgcagctgagccagcagctgtctggaatcaacgccgtgttctactacagcaccagcatcttcgagaaggccggcgtgcagcagcctgtgtatgccacaatcggcagcggcatcgtgaacaccgcctttaccgtggtgtccctgtttgtggtggaaagagccggcagaagaaccctgcacctgattggactggccggcatggccggatgtgccattctgatgacaatcgccctggctctgctggaacagctgccctggatgagctacctgagcatcgtggccattttcggcttcgtggccttctttgaagtcggccctggacctattccttggtttatcgtggccgagctgttttctcagggccccagacctgctgctattgccgtggccggattcagcaactggaccagcaatttcatcgtgggcatgtgcttccagtacgtcgagcagctgtgtggcccctacgtgttcatcattttcaccgtgctgctggtcctgttcttcatcttcacctacttcaaggtgcccgagacaaagggcagaaccttcgacgagatcgccagcggctttagacaaggcggcgctagccagagcgataagacccctgaggaactgtttcaccctctgggcgccgattctcaggtgtga

SEQ ID NO: 6 - DNA, 1491 bp, Homo sapiens
atggggacccagaaagtgacccctgctctgatcttcgccatcaccgtggccaccatcggcagcttccagttcggctataacaccggcgtgatcaacgcccctgagaagatcatcaaagagttcatcaacaagaccctgaccgacaagggcaacgccccaccttctgaagtgctgctgacaagcctgtggtccctgagcgtggccatcttttctgtcggcggcatgatcggaagcttcagcgtgggcctgttcgtgaacagattcggcagacggaactccatgctgatcgtgaacctgctggctgtgaccggcggctgttttatgggactgtgcaaggtggccaagagcgtggaaatgctgatcctgggcagactggtcatcggcctgttctgtggcctgtgcaccggcttcgtgcctatgtacatcggcgagatcagccccacagctctgagaggcgctttcggcacactgaaccagctgggcatcgttgtgggaatcctggtggcccagatcttcggcctggaattcatcctgggctccgaggaactgtggcctctgctgctgggctttacaatcctgcctgccatcctgcagtctgccgctctgcctttctgtcctgagagccccagattcctgctgatcaaccggaaagaggaagagaacgccaagcagatcctgcagcggctgtggggaacacaggatgtgtcccaggacatccaagagatgaaggacgagagcgcccggatgagccaagagaagcaagtgaccgtgctggaactgttccgggtgtccagctacagacagcccatcatcatcagcatcgtgctgcagctgagccagcagctgtccggaatcaacgccgtgttctactacagcaccggcatcttcaaggacgccggggtgcaagagcctatctacgccacaattggagccggcgtcgtgaacaccatctttaccgtggtgtccctgttcctggtggaaagagctgggagaagaaccctgcacatgatcggccttggcggaatggccttctgcagcaccctgatgacagtgtccctgctgctgaaggacaactacaacggcatgagcttcgtgtgcatcggcgccatcctggtgttcgtggccttctttgagatcggccctggacctattccttggtttatcgtggccgagctgttctctcagggccctagacctgctgctatggccgtggccggatgtagcaactggaccagcaatttcctcgtgggactgctgttccctagcgccgctcattatctgggcgcctacgtgttcatcatcttcaccggctttctgatcaccttcctggccttcaccttcttcaaggtgcccgagacacggggcagaaccttcgaggatatcacccgggcctttgaaggacaggctcacggcgctgatagaagcggaaaggacggcgtgatggaaatgaacagcatcgagcccgccaaagaaaccaccaccaacgtgtga

SEQ ID NO: 7 - DNA, 1479 bp, Mus musculus
atggatcccagcagcaagaaggtgacgggccgcctcatgttggctgtgggaggagcagtgctcggatcactgcagttcggctataacactggtgtcatcaacgccccccagaaggttattgaggagttctacaatcaaacatggaaccaccgctacggagagcccatcccatccaccacactcaccacgctttggtctctctccgtggccatcttctctgtcgggggcatgattggttccttctctgtcggcctctttgttaatcgctttggcaggcggaactccatgctgatgatgaacctgttggcctttgtggctgctgtgcttatgggcttctccaaactgggcaagtcctttgagatgctgatcctgggccgcttcatcatcggtgtgtactgcggcctgactactggctttgtgcccatgtatgtgggagaggtgtcacctacagctctacgtggagccctaggcacactgcaccagctgggaatcgtcgttggcatccttattgcccaggtgtttggcttagactccatcatgggcaatgcagacttgtggcctctgctgctcagtgtcatcttcatcccagccctgctacagtgtatcctgttgcccttctgccccgagagcccccgcttcctgctcatcaatcgtaacgaggagaaccgggccaagagtgtgctgaagaagcttcgagggacagccgatgtgacccgagacctgcaggagatgaaagaagagggtcggcagatgatgcgggagaagaaggtcaccatcttggagctgttccgctcacccgcctaccgccagcccatcctcatcgctgtggtgctgcagctgtcccagcagctgtcgggtatcaatgctgtgttctactactcaacgagcatcttcgagaaggcaggtgtgcagcagcctgtgtacgccaccatcggctccggtatcgtcaacacggccttcactgtggtgtcgctgtttgttgtagagcgagctggacgacggaccctgcacctcattggcctggctggcatggcaggctgtgctgtgctcatgaccatcgccctggccttgctggaacggctgccttggatgtcctatctgagcatcgtggccatctttggctttgtggccttctttgaagtaggccctggtcctattccatggttcattgtggccgagctgttcagccaggggccccgtcctgctgctattgctgtggctggcttctccaactggacctcaaacttcattgtgggcatgtgcttccagtatgtggagcaactgtgcggcccctacgtcttcatcatcttcacggtgctcctcgtgctcttcttcatcttcacctacttcaaagtccctgagaccaaaggccgaaccttcgatgagatcgcttccggcttccggcaggggggtgccagccaaagtgacaagacacccgaggagctcttccaccctctgggggcggactcccaagtgtga

SEQ ID NO: 8 - DNA, 1482 bp, Mus musculus
atggggacaacgaaggtgaccccatctctggtgttcgccgtgactgttgccacgatcggctctttccagtttggctacaacactggagtcatcaatgcacctgagacaatcctaaaggactttcttaactacactttggaagagcggttagaagacctaccaagtgagggactgctgactgccctctggtccttatgtgtggccatcttctctgttggtggcatgattggctctttttctgttggactctttgtcaaccgctttggcagacgcaactctatgcttctagtcaacttgctggccatcattgcgggctgccttatgggattcgccaagatagcggagtctgttgaaatgctgatcctgggccgcttactcattggcattttctgtggcctgtgcacgggctttgtgcctatgtacattggagaggtgtctcccactgcccttcggggtgcatttggcacactaaaccagctgggcatcgttgttgggattctggtagctcagatctttggtttggactttattctgggctctgaagaactgtggcctgggctccttggcttaaccatcattccagctatcctgcaaagcgcagcccttccattttgccctgagagtccaagattcttgctcattaacaaaaaggaggaagaccaagctacagagatcctgcagcgcttgtggggcacctcggacgtggtccaggagatccaggagatgaaggatgagagtgttcggatgtcacaggagaagcaggtgactgtgctggagctcttcaggtcacccaactacgtccagccgcttctcatctccattgtcctccagctgtctcagcagctctctgggatcaatgctgtgttctattactcaacaggaatcttcaaggacgcgggtgtccaggaaccgatctatgccacgattggagcaggcgtggtcaatactatcttcactgtagtttctctgttcctggtggagagggcagggaggagaaccctgcatatgataggcctgggaggcatggctgtttgctccgttttcatgacgatttcgctgttactaaaggatgactatgaagccatgagctttgtctgtattgtggctatcttgatctacgtagccttctttgagattggacctggccccattccctggtttattgtggctgagctcttcagccagggcccccgcccagctgccattgcggtggctggctgttgtaactggacctccaactttctggtcggaatgctcttcccctcagctgcagcctacttaggagcctacgtttttatcatcttcgctgccttcctcatcttcttcctaatcttcaccttcttcaaagtcccggagaccaaaggcaggactttcgaggacattgcccgggccttcgaggggcaggcgcactctggaaaaggccctgccggtgtggagttgaacagcatgcagccggtcaaggagacccctggcaacgcctga